(19) United States
(12) Patent Application Publication
(10) Pub. No.: US 2022/0364177 A1
(43) Pub. Date: Nov. 17, 2022

(54) DNA MARKERS FOR DIFFERENTIATION OF BIOPSY SAMPLES

(71) Applicant: The Johns Hopkins University, Baltimore, MD (US)

(72) Inventors: Saraswati V. Sukumar, Columbia, MD (US); Mary Jo Fackler, Hunt Valley, MD (US); Bradley Downs, Omaha, NE (US)

(21) Appl. No.: 17/621,122

(22) PCT Filed: Jun. 18, 2020
(86) PCT No.: PCT/US2020/038362

§ 371 Date: Dec. 20, 2021

Related U.S. Application Data
(60) Provisional application No. 62/864,417, filed on Jun. 20, 2019.

(51) Int. Cl. C12Q 1/6886

(52) U.S. Cl. CPC C12Q 1/6886; C12Q 1/6827

(57) ABSTRACT

In accordance with some embodiments, the present invention comprises use of a rapid and accurate QM-MSP and cMethDNA methylation marker-based assays to quickly distinguish between cancer and benign/normal tissues in a biological sample from a subject suspected of having cancer. Methods for detecting breast, colon, and lung cancers in biological samples of suspect tissues and fluids are also provided to assist in triaging subjects suspected of having cancer for expedited biopsy and pathology review in low resource settings.

### REFERENCE TO RELATED APPLICATIONS

This application claims the benefit of U.S. Provisional Patent Application No. 62/864,417, filed on Jun. 20, 2019, which is hereby incorporated by reference for all purposes as if fully set forth herein.

### BACKGROUND OF THE INVENTION

In developing countries of the world, breast cancer is the most frequently diagnosed cancer in women and the leading cause of cancer death, followed by lung and colon (1, 2). Breast cancer incidence is rising globally because of longer life expectancies, decreased burden of infectious diseases, and changes in reproductive risk factors (3-5). Decreases in overall cancer mortality in the U.S. and Canada are associated with advances in screening and in adjuvant therapy (6, 7). Decreases in colon cancer in the western world has been attributed to regimented screening by colonoscopy, stool tests, CT scan etc. (8). Lung cancer screening is offered to adults ages 55 through 77 years who are current or former smokers and are candidates for lung cancer screening based on pack-years of exposure and their general health status (8).

Low resource regions throughout the world have poor breast, as well as colon and lung cancer survival rates partly because of lack of organized screening programs and lack of means to render timely diagnoses (1, 5). To reduce the global burden of breast, colon and lung cancer, we require novel approaches to expedite definitive diagnosis and access to treatment (8). There is a critical shortage of clinical pathologists and resources to perform the complex histopathologic evaluation of cancer biopsies (11, 12). Currently, even after the breast or lung or colon lesion is biopsied, there may be long delays. Therefore, developing a molecular test for quick and accurate differentiation between cancer and non-cancer in a suspicious lesion is important. Breast cancer can be detected by palpation, mammography or ultrasound imaging, lung cancer can be detected by a cough that does not subside or difficulty in breathing, followed by CT scans, and colon cancer can be detected because of appearance of blood in the stool, irregularity in bowel movement, or by colonoscopy. Determining whether the lesion is benign or malignant has important clinical implications, as this will triage the patients requiring urgent diagnosis.

Hypermethylation of CpG islands located in the promoter regions of tumor suppressor genes is recognized as one of the earliest, most frequent, and robust alteration in cancer development (13, 14). However, low-level methylation occurs in certain CpG regions in benign conditions (15-20). Relevant to our study, Euhus et al. performed a genome-wide search for differentially methylated markers in cancer and benign breast epithelial cell lines treated with 5-azacytidine. A select number of markers were evaluated in fine needle aspirations (FNAs) of 97 cancer and 327 benign disease cases. Significant differential methylation was reported for CPNE8, PSAT1, CXCL14, and CLDN1 and GNE (15). However, no further validation of these markers was performed. Other researchers have reported modest differences for methylation markers in serum that failed to provide sufficient ability to distinguish between cancer and benign/normal controls (21, 22).

Liang et al. published a paper on methylation marker based detection of lung cancer using circulating cell free DNA in plasma and showed that their assay is highly sensitive towards early-stage lung cancer, with a sensitivity of 75.0% (55.0%-90.0%) in 20 stage Ia lung cancer patients and 85.7% (57.1%-100.0%) in 7 stage Ib lung cancer patients. The diagnostic test is a sensitive blood based non-invasive diagnostic assay for detecting early stage lung cancer as well as differentiating lung cancers from benign pulmonary nodules. However, it is based on nucleotide sequencing of bisulfite converted DNA which is expensive and requires sophisticated instrumentation and skilled researchers. (Theranostics. 2019 Apr 6;9(7):2056-2070. doi: 10.7150/thno.28119. eCollection 2019). In yet another study a methylation panel of 6 genes (CD01, HOXA9, AJAP1, PTGDR, UNCX, and MARCH11) was selected from TCGA dataset. Promoter methylation of the gene panel was detected in 92.2% (83/90) of the training cohort with a specificity of 72.0% (18/25) and in 93.0% (40/43) of an independent cohort of stage IA primary non small cell lung cancer (NSCLC). In serum samples from the later 43 stage IA subjects and population-matched 42 control subjects, the gene panel yielded a sensitivity of 72.1% (31/41) and specificity of 71.4% (30/42). (Clin. Cancer Res. 2017 Nov. 15;23(22):7141-7152. doi: 10.1158/1078-0432.CCR-17-1222. Epub 2017 Aug. 29). The downside of these studies is that no follow up studies have been performed to validate the markers in a clinical setting. Also, the assay requires considerable amount of DNA since each gene is analyzed separately. Assays of such sophistication are difficult to implement in underdeveloped countries.

For colon cancer as well, methylation markers have been investigated for early detection. Recently, a retrospective analysis using bisulfite pyrosequencing of an 11 marker panel (SFRP1, SFRP2, SRP4, SRP5, WIF1, TUBB6, SOX7, APC1A, APC2, MINT1, RUNX3) in samples from 35 patients with cancer, 78 with dysplasia and 343 without neoplasia undergoing surveillance for UC associated neoplasia across 6 medical centers. For neoplastic mucosa a five marker panel (SFRP2, SFRP4, WIF1, APC1A, APC2) was accurate in detecting pre-cancerous and invasive neoplasia (AUC=0.83; 95% CI: 0.79, 0.88), and dysplasia (AUC=0.88; (0.84, 0.91). For non-neoplastic mucosa a four marker panel (APC1A, SFRP4, SFRP5, SOX7) had modest accuracy (AUC=0.68; 95% CI: 0.62, 0.73) in predicting associated bowel neoplasia through the methylation signature of distant non-neoplastic colonic mucosa. More accurate is a test called CancerSEEK, performed in patients with nonmetastatic, clinically detected cancers of the colorectum, lung, or breast. The specificity of CancerSEEK was greater than 99%: only 7 of 812 healthy controls scored positive. Seventy percent of colorectal, 60% lung and about 30% of breast cancers were detectable by this test. (Science, 2018 Feb. 23;359(6378):926-930. doi: 10.1126/science.aar3247. Epub 2018 Jan. 18).

There are also similar issues such as lack of screening, lack of pathologists, and lack of reliable testing that plague subjects afflicted with other cancers in low and middle income countries of the world, including cancers such as colon and lung cancers.

Thus, there still exists an unmet need for better markers for breast, lung and colon cancers, as well as simple on site diagnosis of various types of clinical samples, especially in the developing world.

### SUMMARY OF THE INVENTION

In accordance with the embodiments set forth in the present application, the present inventors provide the selection of a panel of DNA methylation markers that distinguishes between normal/benign and cancer breast, colon and lung in biological samples with high sensitivity and specificity using the QM-MSP method, and its variant, the cMethDNA method. The results described herein show that the different panels of selected genes and the assays have potential to detect breast cancer in samples (including fine needle aspirates, for example) taken from suspicious breast lesions identified by mammography, as well as clinical samples from colon and lung lesions. It will be understood by those of ordinary skill in the art, that the inventive methods disclosed herein can be applied to any DNA of any biological sample, including cells, tissues from the primary lesion, lymph node or distant metastatic sites or fluids, such as ductal fluid, nipple fluid, sputum, feces, urine, blood or circulating cell-free DNA. This has the potential for rapid diagnosis in poor resource countries.

As such, in accordance with an embodiment, the present invention provides a method for detecting the presence of one, two, or more methylated gene regions in a biological sample of breast tissue from a subject suspected of having breast cancer comprising: a) hybridizing nucleic acid obtained from the sample with two or more QM-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671; b) performing QM-MSP on the breast tissue sample from a); and c) detecting if any of the specific CpG regions of the one, two, or more genes of the suspicious breast tissue sample of a) are hypermethylated compared to the level of methylation of a normal/benign breast tissue sample.

In accordance with another embodiment, the present invention provides a method for triaging a subject with one or more suspicious lesions in the breast into biopsy and pathological review comprising: a) hybridizing nucleic acid obtained from a biological sample of breast tissue from the suspicious lesions in the breast of the subject with two or more QM-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671; b) performing QM-MSP on the breast tissue sample from a); c) detecting if any of the specific CpG regions of one, two, or more genes of the suspicious breast tissue of a) are hypermethylated compared to the level of methylation of normal/benign breast tissue sample; and d) triaging the subject into biopsy of the suspicious lesion and pathological review when the specific CpG regions of one, two, or more of the genes of b) are hypermethylated compared to the level of methylation of a normal/benign breast tissue sample.

In accordance with yet another embodiment, the present invention provides a method for detecting the presence of one, two, or more methylated gene regions in a biological sample from a suspicious colon lesion from a subject comprising: a) hybridizing nucleic acid obtained from the sample of the suspicious colon lesion from the subject with two or more QM-MSP primer and probe sets specific for the genes selected from the group consisting of AKR1B1, APC, ARHGEF7, COL6A2, GAS7C, GPX7, HIN1, HIST1H3C, MAL, TM6SF1, TMEFF2, TWIST1, and ZNF671; b) performing QM-MSP on the suspicious colon lesion sample from a); and c) detecting if any of the specific CpG regions of the one, two, or more genes of the suspicious colon lesion of a) are hypermethylated compared to the level of methylation of a normal/benign colon tissue sample.

In accordance with another embodiment, the present invention provides a method for triaging a subject with one or more suspicious colon lesions into biopsy and pathological review comprising: a) hybridizing nucleic acid obtained from a biological sample from a suspicious colon lesion from the subject with two or more QM-MSP primer and probe sets specific for the genes selected from the group consisting of AKR1B1, APC, ARHGEF7, COL6A2, GAS7C, GPX7, HIN1, HIST1H3C, MAL, TM6SF1, TMEFF2, TWIST1, and ZNF671; b) performing QM-MSP on the biological sample from a suspicious colon lesion from the subject of a); c) detecting if any of the specific CpG regions of the one, two, or more genes from a suspicious colon lesion from the subject of a) are hypermethylated compared to the level of methylation of normal/benign colon tissue; and d) triaging the subject into colon biopsy of the suspicious lesion and pathological review when the specific CpG regions of one, two, or more of the genes of b) are hypermethylated compared to the level of methylation of a normal/benign colon tissue sample.

In accordance with a further embodiment, the present invention provides a method for detecting the presence of one, two, or more methylated gene regions in a biological sample from a suspicious lung lesion from a subject comprising: a) hybridizing nucleic acid obtained from the sample of suspicious lung lesion from the subject with two or more QM-MSP primer and probe sets specific for the genes selected from the group consisting of APC, ARHGEF7, CCND2, COL6A2, HIST1H3C, HOXB4, RARB, RASGRF2, RASSF1, TM6SF1, TWIST1, and ZNF671; b) performing QM-MSP on the sample of suspicious lung lesion from the subject from a); and c) detecting if any of the specific CpG regions of the one, two, or more genes from the sample of suspicious lung lesion from the subject of a) are hypermethylated compared to the level of methylation of normal/benign lung tissue.

In accordance with still another embodiment, the present invention provides a method for triaging a subject with one or more suspicious lung lesions into biopsy and pathological review comprising: a) hybridizing nucleic acid obtained from a sample from the subject with two or more QM-MSP primer and probe sets specific for the genes selected from the group consisting of APC, ARHGEF7, CCND2, COL6A2, HIST1H3C, HOXB4, RARB, RASGRF2, RASSF1, TM6SF1, TWIST1, and ZNF671; b) performing QM-MSP on the sample from a); c) detecting if any of the specific CpG regions of the one, two, or more genes of a) are hypermethylated compared to the level of methylation of normal/benign lung tissue; and d) triaging the subject into lung biopsy of the suspicious lesion and pathological review when the specific CpG regions of one, two, or more of the genes of b) are hypermethylated.

### BRIEF DESCRIPTION OF THE DRAWINGS

FIG. 1. FFPE, formalin fixed paraffin embedded; IDC, invasive ductal carcinoma; ILC, invasive lobular carcinoma; DCIS, ductal carcinoma in situ; QM-MSP, quantitative multiplex methylation-specific PCR; AUC, area under the ROC curve; FNA, fine needle aspirate. Total FFPE samples: n=472 (449 randomized to test and training Sets+23 ILC added to test set).

FIG. 2. Performance of the 10 individual gene markers in a training set. Box-Whiskers plots (Tukey method) depict the percent methylation (Y-axis) for each of the 10-gene markers in FFPE tissues obtained from patients with IDC/DCIS versus Benign/Normal. Percent methylation in the benign/normal tissues, sub-classified according to histologic types is shown. Mann-Whitney P-values are indicated. Sample number (N) is shown below the X-axis. IDC, invasive ductal carcinoma; DCIS, ductal carcinoma in situ; Fibro, fibroadenoma; Pap, papilloma; UDH, usual ductal hyperplasia.

FIGS. 3A-3B. Performance of the 10-gene marker panel to distinguish between benign and cancer tissue. FFPE samples of IDC/DCIS and Benign/Normal tissues were assayed by QM-MSP. Histogram plots indicate the percent methylation (colored segment) and cumulative methylation (bar height, Y-axis) for each of the 10-gene markers in the panel. Insets: 1) Box plots show the median cumulative methylation of cancer (IDC/DCIS) versus benign/normal tissues. 2) ROC analyses indicate the discriminatory power of the 10-gene marker panel. 3A. Training set. The laboratory threshold that provided highest sensitivity and specificity for detection of cancer in the training cohort was 14.5 CMI, based on ROC analysis. 3B. Test set. Conditions established in the training set were locked and performance of the test set was evaluated as in 3A. Insets: 1) Box plots show the median cumulative methylation of cancer (IDC/DCIS) versus benign/normal tissues. 2) ROC analyses indicate the discriminatory power of the 10-gene marker panel. The dashed line indicates our target sensitivity at 90%, and dot indicates the cutoff at 14.5 CMI units on the curve. CMI, cumulative methylation index; IDC, invasive ductal carcinoma; ILC, invasive lobular carcinoma, DCIS, ductal carcinoma in situ; FA, fibroadenoma; Pap, papilloma; UDH, usual ductal hyperplasia, and cysts. ROC, receiver operator characteristic; AUC, area under the ROC curve. Mann-Whitney P-values are indicated.

FIG. 4. Evaluation of the 25 breast cancer-associated genes. The percent (%) methylation for 25 individual breast cancer-associated genes was determined using the QM-MSP assay in the marker selection cohort. Scatter plots depict gene methylation intensity (Y-axis, % methylation) in cancer [invasive ductal carcinomas (IDC) and ductal carcinoma in situ (DCIS)] versus Benign/Normal samples (X-axis; legend). Mann-Whitney P-values where significant (<0.01) are shown.

FIGS. 5A-5C. Evaluation of Differences in Gene Methylation by Immunohistochemical (IHC) Subtype and Country of Origin. 5A. Methylation by IHC Subtype. DNA methylation in ER/PR+, HER2−(red), ER/PR+, HER2+(blue), ER/PR−,HER+(fushcia), ER/PR−, HER2−(green). 5B. Methylation by Region. DNA methylation in breast cancer from the U.S. (red), China (blue), and S. Africa (green). For 5A) and 5B), box plots indicate the levels of cumulative methylation (CMI) of the 10-gene marker panel and scatter plots indicate % methylation of individual genes as assessed by QM-MSP. For multiple comparison between groups the Kruskal-Wallis test, unadjusted P-values are indicated (*). N=number of samples. 5C. Performance of the 10-gene panel (as measured by area under the ROC curve) assessed separately in tissues from the U.S., China and Africa. The dashed line indicates our target sensitivity at 90%, and dots indicate the cutoff at 14.5 CMI units on each curve. Sensitivity, specificity and AUC values with 95% CI are shown in the table.

FIGS. 6A-6B depict the cumulative methylation of a 13-gene marker panel evaluated in colon cancer tissues. Slides consisting of sections of formalin fixed paraffin embedded tissue were macrodissected, processed and QM-MSP was performed to determine the extent of DNA methylation within the sample. The histogram displays the cumulative methylation level of the panel, as well as the percent methylation of each gene (colored segment). Colon Study #1 (FIG. 6A) consisted of 8 cancers, 8 benign adenomas and 6 normal tissues located adjacent to cancer/adenoma. Colon Study #2 (FIG. 6B) consisted of 17 cancers, 11 adenomas and 9 normal adjacent tissues.

FIG. 7 depicts the cumulative methylation of a 12-gene marker panel evaluated in lung cancer. Slides of formalin fixed paraffin embedded tissue sections were scraped, processed and QM-MSP was performed to determine the extent of DNA methylation in the sample. The histogram displays the cumulative methylation level of the marker panel, as well as the percent methylation of each gene (colored segment) for 134 lung adenocarcinoma and 16 normal tissues adjacent to tumor.

### DETAILED DESCRIPTION OF THE INVENTION

The genomes of higher eukaryotes contain the modified nucleoside 5-methyl cytosine (5-meC). This modification is usually found as part of the dinucleotide CpG in which cytosine is converted to 5-methylcytosine in a reaction that involves flipping a target cytosine out of an intact double helix and transfer of a methyl group from S-adenosylmethionine by a methyltransferase enzyme (31). This enzymatic conversion is the primary epigenetic modification of DNA known to exist in vertebrates and is essential for normal embryonic development (32-34).

In eukaryotes, DNA methylation regulates normal cellular processes such as genomic imprinting, chromosomal instability, and X-chromosome inactivation. Typically, DNA methylation occurs at the fifth carbon position of cytosine at dinucleotide 5′-CpG-3′ sites in or near gene promoters termed CpG islands or shores. Methylation controls gene expression by down-regulating transcription either by directly inhibiting transcriptional machinery or indirectly through the recruitment of chromatin remodeling proteins. Chromosomal methylation patterns change dynamically during embryonic development, and the correct methylation patterns have to be maintained throughout an individual's lifetime. Changes in methylation patterns are linked to aging, and errors in DNA methylation are among the earliest changes that occur during oncogenesis. Thus, the detection of methylation at gene promoters is important for diagnosing and/or monitoring patients with cancer.

Epigenetic alterations, including DNA methylation, interrupt the DNA-RNA-protein axis which describes how genetic information is transcribed into messenger RNAs (mRNAs). The correlation between genomic DNA variation, mRNA copy numbers and protein levels may be described by DNA methylation levels. Thus, co-measurement of DNA methylation levels and corresponding down-stream mRNA levels can be important to understanding the mechanism of epigenetic cellular regulation.

Several methods have been developed to detect and quantify methylation efficiently and accurately. The most common technique is the bisulfite conversion method, which converts unmethylated cytosines to uracil. Once converted, the methylation profile of DNA can be determined by standard PCR techniques, QM-MSP PCR methods (U.S. Pat. No. 9,416,404), and the like.

The present invention is based on a methylated gene marker panel having between 2 and 20 gene regions that easily distinguishes between benign breast, colon and lung tissues and cancerous tissues with high levels of sensitivity and specificity. In one embodiment, the gene panel is a 10-gene panel. In alternative embodiments, the gene panel is a 1, 2, 3, 4, 5, 6, 7, 8, 9, 10, 11, 12, 13, 14, 15, 16, 17, 18, 19, and 20 gene panel, as well as permutations thereof.

Thus, in one aspect, the present inventors have demonstrated the utility of assessing methylated markers in a biological or clinical sample from a suspected lesion of the breast. For example, biological samples can be taken from the breast, including, for example, from ductal lavage/ductoscopy fluids and cells, nipple fluids, and fine needle aspirates as well as tissues and core biopsies. The inventive assays use a panel of methylated gene markers for breast cancer screening in subjects with suspicious lesions, including those detected by palpation, mammography or ultrasound imaging. The assays of the present invention are elegant, as they are simple in their execution and thus could be mobilized in underserved regions of the world for the discrimination of suspicious breast mammograms or lesions, from actual breast cancer or benign lesions.

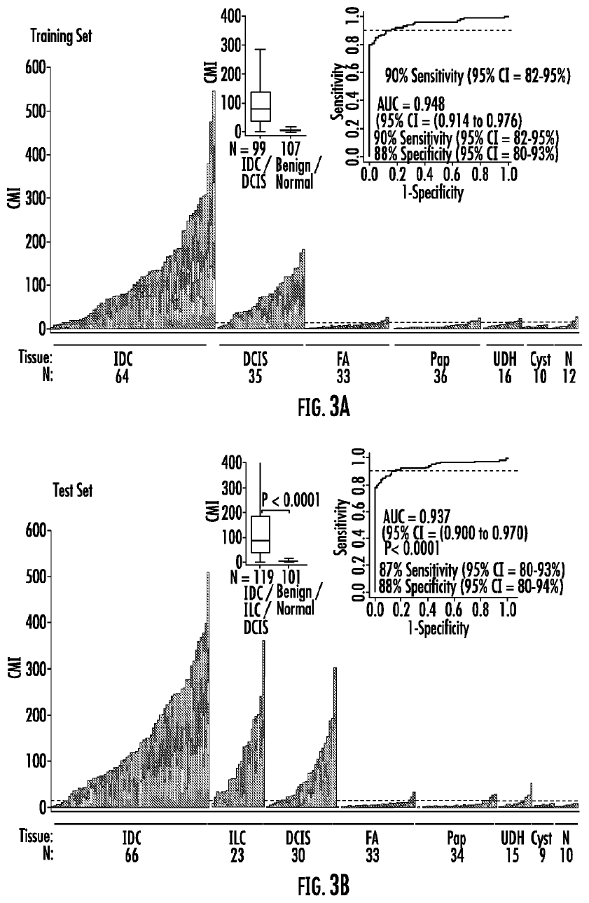

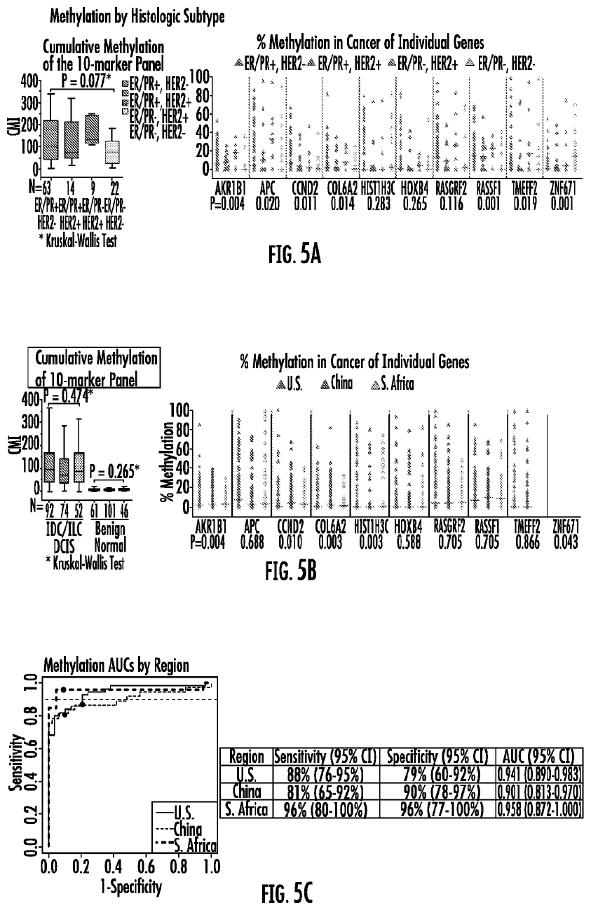

In accordance with multiple embodiments, the multi-gene panel assays of the present invention are broadly applicable to subjects, including subjects from diverse ethnicities and world regions, as no significant differences in cumulative methylation of the 10-gene marker and greater panel between samples from the United States, China or South Africa were found. The invention assay data and outcomes attest to the strength of using a multi-gene panel of markers rather than a single gene marker (FIGS. 3 and 5).

It will be understood by those of ordinary skill in the art that the assays and methods provided herein are taken from subjects for a variety of clinical reasons including, for example, to monitor disease or progression of disease, detect disease recurrence or monitor effectiveness of a treatment regimen.

Therefore, in accordance with a first embodiment, the present invention provides a method for detecting the presence of one, two, or more methylated gene regions in a biological sample of breast tissue from a subject suspected of having breast cancer comprising: a) hybridizing nucleic acid obtained from the sample with two or more QM-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671; b) performing QM-MSP on the breast tissue sample from a); and c) detecting if any of the specific CpG regions of the one, two, or more genes of the breast tissue sample of a) are hypermethylated compared to the level of methylation of a normal/benign breast tissue sample.

In accordance with a second embodiment, the present invention provides a method for triaging a subject with one or more suspicious lesions in the breast into biopsy and pathological review comprising: a) hybridizing nucleic acid obtained from a biological sample of breast tissue from the suspicious lesions in the breast of the subject with two or more QM-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671; b) performing QM-MSP on the breast tissue sample from a); c) detecting if any of the specific CpG regions of the one, two, or more genes of the breast tissue of a) are methylated compared to the level of methylation of normal/benign breast tissue sample; and d) triaging the subject into biopsy of the suspicious lesion and pathological review when the specific CpG regions of one, two, or more of the genes of b) are hypermethylated compared to the level of methylation of a normal/benign breast tissue sample.

In accordance with a third embodiment, the present invention provides a method for monitoring disease or progression of disease in a subject having or suspected of having breast cancer comprising: a) hybridizing nucleic acid obtained from a biological sample of breast tissue from the suspicious lesions in the breast of the subject with two or more QM-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671; b) performing QM-MSP on the breast tissue sample from a); c) detecting if any of the specific CpG regions of the one, two, or more genes of the lymph node sample of a) are hypermethylated compared to the level of methylation of a previous sample of breast tissue from the subject and, d) triaging the subject into further or different treatment when any of the specific CpG regions of the one, two, or more genes of the sample of breast tissue of a) are hypermethylated compared to the level of methylation of the previous sample of breast tissue from the subject.

In accordance with a fourth embodiment, the present invention provides a method for detecting disease recurrence in a subject undergoing treatment or having been treated for breast cancer comprising: a) hybridizing nucleic acid obtained from a biological sample of breast tissue from the suspicious lesions in the breast of the subject with two or more QM-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671; b) performing QM-MSP on the breast tissue sample from a); c) detecting if any of the specific CpG regions of the one, two, or more genes of the biological sample of breast tissue of a) are hypermethylated compared to the level of methylation of a previous biological sample of breast tissue from the subject; and, d) triaging the subject into a change in treatment when any of the specific CpG regions of one, two, or more genes of the biological sample of breast tissue of a) are hypermethylated compared to the level of methylation of the previous biological sample of breast tissue from the subject.

In some embodiments, the methods for detection of breast cancer can detect increased methylation of 3, 4, 5, 6, 7, 8, 9, or 10, and up to 20 sets of different specific CpG regions of genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671, as well as permutations thereof.

As used herein, the term “subject” refers to a human subject. Typically, the subject will be a human female, however the methods, especially those referring to colon and lung cancers, are not limited to samples from females.

In a second aspect, the present inventors have demonstrated the utility of assessing methylated markers in a suspected colon lesion, including samples from stool, and blood, as well as tissues and core biopsies, using a panel of methylated gene markers for colon cancer screening in subjects with suspicious lesions, including those detected by colonoscopy, blood or stool samples, and thus could be mobilized in underserved regions of the world for the discrimination of suspicious colon polyps, blood or stool, from actual colon cancer or benign/normal lesions.

Biological samples can be taken from colon tissue, including, for example, samples from polyps or stool, or blood and biological samples can be taken from lung tissue, including, for example, biopsy or sputum and blood.

As used herein, the term “biopsy” can be any medical or clinical procedure where a sample or samples comprising cells from the tissue of a subject are removed for analysis. This can include breast core biopsy, colonoscopy biopsy, or lung biopsies.

Therefore, in accordance with a fifth embodiment, the present invention provides a method for detecting the presence of two or more methylated gene regions in a biological sample from a suspicious colon lesion from a subject comprising: a) hybridizing nucleic acid obtained from a sample of tissue, cells, stool, sigmoidoscopy or endoscopy-irrigation-derived cells, saliva, blood or urine from the subject with two or more QM-MSP primers and probes specific for the genes selected from the group consisting of AKR1B1, APC, ARHGEF7, COL6A2, GAS7C, GPX7, HIN1, HIST1H3C, MAL, TM6SF1, TMEFF2, TWIST1, and ZNF671; b) performing QM-MSP on the sample from a); and c) detecting if any of the specific CpG regions of the one, two, or more genes of a) are hypermethylated compared to the level of methylation of normal/benign colon tissue.

In accordance with a sixth embodiment, the present invention provides a method for triaging a subject with one or more suspicious colon lesions into biopsy and pathological review comprising: a) hybridizing nucleic acid obtained from a sample of tissue, cells, stool, sigmoidoscopy or endoscopy-irrigation-derived cells, saliva, blood or urine from the subject with two or more QM-MSP primers and probes specific for the genes selected from the group consisting of AKR1B1, APC, ARHGEF7, COL6A2, GAS7C, GPX7, HIN1, HIST1H3C, MAL, TM6SF1, TMEFF2, TWIST1, and ZNF671; b) performing QM-MSP on the sample from a); c) detecting if any of the specific CpG regions of the one, two, or more genes of a) are hypermethylated compared to the level of methylation of normal/benign colon tissue; and d) triaging the subject into colon biopsy of the suspicious lesion and pathological review when the specific CpG regions of one, two, or more of the genes of b) are hypermethylated.

In accordance with a seventh embodiment, the present invention provides a method for monitoring disease or progression of disease in a subject having or suspected of having colon cancer comprising: a) hybridizing nucleic acid obtained from a biological sample of colon tissue from the suspicious lesions in the colon of the subject with two or more QM-MSP primer and probe sets specific for the genes selected from the group consisting of AKR1B1, APC, ARHGEF7, COL6A2, GAS7C, GPX7, HIN1, HIST1H3C, MAL, TM6SF1, TMEFF2, TWIST1, and ZNF671; b) performing QM-MSP on the sample from a); c) detecting if any of the specific CpG regions of the one, two, or more genes of a) are methylated compared to the level of methylation of normal/benign colon tissue; and d) triaging the subject into further or different treatment when any of the specific CpG regions of the one, two, or more genes of the sample of colon tissue of a) are hypermethylated compared to the level of methylation of the previous sample of colon tissue from the subject.

In accordance with a eighth embodiment, the present invention provides a method for detecting disease recurrence in a subject undergoing treatment or having been treated for colon cancer comprising: a) hybridizing nucleic acid obtained from a biological sample of colon tissue from the suspicious lesions in the colon of the subject with two or more QM-MSP primer and probe sets specific for the genes selected from the group consisting of AKR1B1, APC, ARHGEF7, COL6A2, GAS7C, GPX7, HIN1, HIST1H3C, MAL, TM6SF1, TMEFF2, TWIST1, and ZNF671; b) performing QM-MSP on the sample from a); c) detecting if any of the specific CpG regions of the one, two, or more genes of a) are hypermethylated compared to the level of methylation of normal/benign colon tissue; and d) triaging the subject into a change in treatment when any of the specific CpG regions of one, two, or more genes of the biological sample of colon tissue of a) are hypermethylated compared to the level of methylation of the previous biological sample of colon tissue from the subject.

In some embodiments, the methods for detection of colon cancer can detect increased methylation 3, 4, 5, 6, 7, 8, 9, or 10 up to 13 different sets of specific CpG regions of genes selected from the group consisting of AKR1B1, APC, ARHGEF7, COL6A2, GAS7C, GPX7, HIN1, HIST1H3C, MAL, TM6SF1, TMEFF2, TWIST1, and ZNF671, as well as permutations thereof.

In another aspect, the present inventors have demonstrated the utility of assessing methylated markers in a suspected lung lesion, including samples from oral gavage, saliva, as well as needle or core biopsies, blood or urine, using a panel of methylated gene markers for lung cancer screening in subjects with suspicious lesions, including those detected by, for example, bronchoscopy, oral gavage, and saliva samples, blood and thus could be mobilized in underserved regions of the world for the screening of suspicious lung polyps, blood or sputum, to distinguish between lung cancer and benign/normal lesions.

Therefore, in accordance with a ninth embodiment, the present invention provides a method for detecting the presence of one, two, or more methylated gene regions in a biological sample from a suspicious lung lesion, including samples from oral gavage, saliva, as well as needle or core biopsies, blood, or urine, using a panel of methylated gene markers for lung cancer screening in subjects with suspicious lesions, comprising: a) hybridizing nucleic acid obtained from a sample of the lesion from the subject with two or more QM-MSP primer and probe sets specific for the genes selected from the group consisting of APC, ARHGEF7, CCND2, COL6A2, HIST1H3C, HOXB4, RARB, RASGRF2, RASSF1, TM6SF1, TWIST1, and ZNF671; b) performing QM-MSP on the sample from a); c) detecting if any of the specific CpG regions of the one, two, or more genes of a) are hypermethylated compared to the level of methylation of normal/benign lung tissue.

In accordance with a tenth embodiment, the present invention provides a method for triaging a subject with one or more suspicious lung lesions into biopsy and pathological review comprising: a) hybridizing nucleic acid obtained from a sample from the subject with two or more QM-MSP primer and probe sets specific for the genes selected from the group consisting of APC, ARHGEF7, CCND2, COL6A2, HIST1H3C, HOXB4, RARB, RASGRF2, RASSF1, TM6SF1, TWIST1, and ZNF671; b) performing QM-MSP on the sample from a); c) detecting if any of the specific CpG regions of the one, two, or more genes of a) are hypermethylated compared to the level of methylation of normal/benign lung tissue; and d) triaging the subject into lung biopsy of the suspicious lesion and pathological review when the specific CpG regions of one, two, or more of the genes of b) are hypermethylated.

In accordance with a eleventh embodiment, the present invention provides a method for monitoring disease or progression of disease in a subject having or suspected of having lung cancer comprising: a) hybridizing nucleic acid obtained from a sample from the subject with two or more QM-MSP primer and probe sets specific for the genes selected from the group consisting of APC, ARHGEF7, CCND2, COL6A2, HIST1H3C, HOXB4, RARB, RASGRF2, RASSF1, TM6SF1, TWIST1, and ZNF671; b) performing QM-MSP on the sample from a); c) detecting if any of the specific CpG regions of the one, two, or more genes of a) are hypermethylated compared to the level of methylation of normal/benign lung tissue; and d) triaging the subject into further or different treatment when any of the specific CpG regions of the one, two, or more genes of the sample of lung tissue of a) are hypermethylated compared to the level of methylation of the previous sample of lung tissue from the subject.

In accordance with a twelfth embodiment, the present invention provides a method for detecting disease recurrence in a subject undergoing treatment or having been treated for lung cancer comprising: a) hybridizing nucleic acid obtained from a sample from the subject with two or more QM-MSP primer and probe sets specific for the genes selected from the group consisting of APC, ARHGEF7, CCND2, COL6A2, HIST1H3C, HOXB4, RARB, RASGRF2, RASSF1, TM6SF1, TWIST1, and ZNF671; b) performing QM-MSP on the sample from a); c) detecting if any of the specific CpG regions of the one, two, or more genes of a) are hypermethylated compared to the level of methylation of normal/benign lung tissue; and d) triaging the subject into a change in treatment when any of the specific CpG regions of one, two, or more genes of the biological sample of lung tissue of a) are hypermethylated compared to the level of methylation of the previous biological sample of lung tissue from the subject.

In some embodiments, the methods for detection of lung cancer can detect increased methylation 3, 4, 5, 6, 7, 8, 9, or 10 up to 12 different sets of specific CpG regions of genes selected from the group consisting of APC, ARHGEF7, CCND2, COL6A2, HIST1H3C, HOXB4, RARB, RASGRF2, RASSF1, TM6SF1, TWIST1, and ZNF671, as well as permutations thereof.

As used herein, the term “QM-MSP primers and probes” means the PCR primers and probes used to identify the methylated CpG regions methylated in the nucleic acid of the sample and disclosed in Table 1.

TABLE 1
Primers Used for Methylation Status

| Primer Name | 5′ to 3′ Sequence |
|---|---|
| AKR1B1_Ext_F | GYGTAATTAATTAGAAGGTTTTTT |
| | (SEQ ID NO: 1) |
| AKR1B1_Ext_R | AACACCTACCTTCCAAATAC |
| | (SEQ ID NO: 2) |
| AKR1B1_FM | GCGCGTTAATCGTAGGCGTTT |
| | (SEQ ID NO: 3) |
| AKR1B1_RM | CCCAATACGATACGACCTTAAC |
| | (SEQ ID NO: 4) |
| AKR1B1_M_ | CGTACCTTTAAATAACCCGTAAAATCGA |
| Probe | (SEQ ID NO: 5) |
| AKR1B1_FUM | TGGTGTGTTAATTGTAGGTGTTTT |
| | (SEQ ID NO: 6) |
| AKR1B1_RUM | CCCAATACAATACAACCTTAACC |
| | (SEQ ID NO: 7) |
| AKR1B1_U_ | ACATACCTTTAAATAACCCATAAAATCAAC |
| Probe | (SEQ ID NO: 8) |
| STD_AKR1B1_F | TTTGTTGATGTTTTGTGGAAGTAAG |
| | (SEQ ID NO: 9) |
| STD_AKR1B1_R | ATTCATCAATACTTTCAAATAACACA |
| | (SEQ ID NO: 10) |
| STD_AKR1B1_ | AAATACATTATCCTACCACTAACAATACA |
| Probe | (SEQ ID NO: 11) |
| APC_Ext_F2 | AAAACCCTATACCCCACTAC |
| | (SEQ ID NO: 12) |
| APC_Ext_R2 | GGTTGTATTAATATAGTTATATGT |
| | (SEQ ID NO: 13) |
| APC_FM | AATACGAACCAAAACGCTCCC |
| | (SEQ ID NO: 14) |
| APC_RM | TATGTCGGTTACGTGCGTTTATAT |
| | (SEQ ID NO: 15) |
| APC_M_Probe | CCCGTCGAAAACCCGCCGATTA |
| | (SEQ ID NO: 16) |
| APC_FUM | TAAATACAAACCAAAACACTCCC |
| | (SEQ ID NO: 17) |
| APC_RUM | GTTATATGTTGGTTATGTGTGTTT |
| | (SEQ ID NO: 18) |
| APC_U_Probe | TTCCCATCAAAAACCCACCAATTAAC |
| | (SEQ ID NO: 19) |
| ARHGEF7_Ext_F | YGTTTYGAGGTGAAGGYGYG |
| | (SEQ ID NO: 20) |
| ARHGEF7_Ext_R | CTCCAACAACCTACAAAAAAC |
| | (SEQ ID NO: 21) |
| ARHGEF7_FM | GTTTTTCGGGTCGTAGCGATG |
| | (SEQ ID NO: 22) |
| ARHGEF7_RM | CAAAAAACCCTCCGAATCCGAA |
| | (SEQ ID NO: ) |
| ARHGEF7_M_ | AAACCACGTAACGATTTACTCGACGA |
| Probe | (SEQ ID NO: 23) |
| ARHGEF7_FUM2 | GTTGTAGTGATGAATTTTGTTGAG |
| | (SEQ ID NO: 24) |
| ARHGEF7_RUM2 | CAAAAAACCCTCCAAATCCAAAAT |
| | (SEQ ID NO: 25) |
| ARHGEF7_U_ | AATAAACCACATAACAATTTACTCAACAAA |
| Probe | (SEQ ID NO: 26) |
| STD_ARHGEF7_F | GGAAATGTGATTTTGATGTTTATGTT |
| | (SEQ ID NO: 27) |
| STD_ARHGEF7_R | ACCCAACACCTATTTCTTAATCAC |
| | (SEQ ID NO: 28) |
| STD_ARHGEF7_ | ACCTACACATCACTAACAAACATATACAA |
| Probe | (SEQ ID NO: 29) |
| CCND2_Ext_F | TATTTTTTGTAAAGATAGTTTTGAT |
| | (SEQ ID NO: 30) |
| CCND2_Ext_R | TACAACTTTCTAAAAAATAACCC |
| | (SEQ ID NO: 31) |
| CCND2_FM | TTTGATTTAAGGATGCGTTAGAGTACG |
| | (SEQ ID NO: 32) |
| CCND2_RM | ACTTTCTCCCTAAAAACCGACTACG |
| | (SEQ ID NO: 33) |
| CCND2_M_Probe | CGCCGCCAACACGATCGACCCTA |
| | (SEQ ID NO: 34) |
| CCND2_FUM2 | TTAAGGATGTGTTAGAGTATGTG |
| | (SEQ ID NO: 35) |
| CCND2_RUM2 | AAACTTTCTCCCTAAAAACCAACTACAAT |
| | (SEQ ID NO: 36) |
| CCND2_U_Probe | AAATCACCACCAACACAATCAACCCTA |
| | (SEQ ID NO: 37) |
| CDKL2_Ext_F | ATGGTTGGGGATAGAGTGAG |
| | (SEQ ID NO: 38) |
| CDKL2_Ext_R | CTACCTAACAAACTAACAATTC |
| | (SEQ ID NO: 39) |
| CDKL2_FM1 | ACGATAGTATGCGGTTCGTGGT |
| | (SEQ ID NO: 40) |
| CDKL2_RM1 | TTCTTCTACGAACGCAATCACGA |
| | (SEQ ID NO: 41) |
| CDKL2_M_Probe | AACCTCGATCCGCCGCAAACTCAC |
| | (SEQ ID NO: 42) |
| CDKL2_FUM1 | GATGATAGTATGTGGTTTGTGGTT |
| | (SEQ ID NO: 43) |
| CDKL2_RUM1 | CACAATCACAAATCCAAAACAAAC |
| | (SEQ ID NO: 44) |
| CDKL2_U_Probe | AACCTCAATCCACCACAAACTCACAC |
| | (SEQ ID NO: 45) |
| STD_CDKL2_F | GGAAGTTGATTATAGATTTGATGTT |
| | (SEQ ID NO: 46) |
| STD_CDKL2_R | ATCACCAAAACACTACAACTAAACA |
| | (SEQ ID NO: 47) |
| STD_CDKL2_Probe | TAAAACTAACAAACCATCACCATCAACC |
| | (SEQ ID NO: 48) |
| COL6A2_Ext_F | AGGTTTAGGAGAAGTTGTAGA |
| | (SEQ ID NO: 49) |
| COL6A2_Ext_R | TACCAACAATAAAAACCCAAAC |
| | (SEQ ID NO: 50) |
| COL6A2_FM | ATTCGGGTTGATAGCGATTCGT |
| | (SEQ ID NO: 51) |
| COL6A2_RM | CGATTCCACCAACGCCCCG |
| | (SEQ ID NO: 52) |
| COL6A2_M_Probe | ATCCCAAAACGAATATAAACGACCCG |
| | (SEQ ID NO: 53) |
| COL6A2_FUM | GATTTGGGTTGATAGTGATTTGTA |
| | (SEQ ID NO: 54) |
| COL6A2_RUM | CAATTCCACCAACACCCCAAC |
| | (SEQ ID NO: 55) |
| COL6A2_U_Probe | ATCCCAAAACAAATATAAACAACCCAAC |
| | (SEQ ID NO: 56) |
| STD_COL6A2_F | TTGGTAAGGTGGTGATGGTGAA |
| | (SEQ ID NO: 57) |
| STD_COL6A2_R | TCTTCTACCATCAACAAACATCC |
| | (SEQ ID NO: 58) |
| STD_COL6A2_ | AACTTTCACCATACAAATAATCACTTCC |
| Probe | (SEQ ID NO: 59) |
| EVI1_Ext_F1 | GGATAGTTTTTTGTYGGGGTA |
| | (SEQ ID NO: 60) |
| EVI1_Ext_R1 | TTTTAACAACCRCACCTTATAC |
| | (SEQ ID NO: 61) |
| EVI1_FM1 | ACGTTGTTAAGTCGGGCGATTT |
| | (SEQ ID NO: 62) |
| EVI1_RM1 | CACCGAAAACCGAACGACCC |
| | (SEQ ID NO: 63) |
| EVI1_M_Probe | AACGTATCCAAACCGCACCGTTTCG |
| | (SEQ ID NO: 64) |
| EVI1_FUM1 | TTATGTTGTTAAGTTGGGTGATTTT |
| | (SEQ ID NO: 65) |
| EVI1_RUM1 | CCACCAAAAACCAAACAACCCA |
| | (SEQ ID NO: 66) |
| EVI1_U_Probe | AAAACATATCCAAACCACACCATTTCAC |
| | (SEQ ID NO: 67) |
| GAS7C_Ext_F | GTAAGGGTTGTTTTTYGGGG |
| | (SEQ ID NO: 68) |
| GAS7C_Ext_R | AACCCTATACCCCTTCTCC |
| | (SEQ ID NO: 69) |
| GAS7C_FM | TAGGTACGCGAGCGTATCGAG |
| | (SEQ ID NO: 70) |
| GAS7C_RM | CCGACGAACTACGTACAATTAC |
| | (SEQ ID NO: 71) |
| GAS7C_M_Probe | TCGTAGTTTCGGTTTTTATAGTTTCGGT |
| | (SEQ ID NO: 72) |
| GAS7C_FUM | TAATAGGTATGTGAGTGTATTGAG |
| | (SEQ ID NO: 73) |
| GAS7C_RUM | TTCCCAACAAACTACATACAATTAC |
| | (SEQ ID NO: 74) |
| GAS7C_U_Probe | TTGTAGTTTTGGTTTTTATAGTTTTGGT |
| | (SEQ ID NO: 75) |
| STD_GAS7C_F | ATGTAGATTGTGGATTTTAGTGTTG |
| | (SEQ ID NO: 76) |
| STD_GAS7C_R | ATACCAACATAATCATCATCACAAAT |
| | (SEQ ID NO: 77) |
| STD_GAS7C_Probe | TTTTGTTATGTATTGGGTGATGTTATTGA |
| | (SEQ ID NO: 78) |
| GPX7_Ext_F | GGTGAAATTGAGGTTTAGAG |
| | (SEQ ID NO: 79) |
| GPX7_Ext_R | ATACTTCTCCAACRACACCA |
| | (SEQ ID NO: 80) |
| GPX7_FM | ACGGTGGTAGCGGCGTGGTT |
| | (SEQ ID NO: 81) |
| GPX7_RM | ACCCCGAATATTAACCGCCTTA |
| | (SEQ ID NO: 82) |
| GPX7_M_Probe | TACTACGCGCAAACCGCAACCCAC |
| | (SEQ ID NO: 83) |
| GPX7_FUM | TGATGGTGGTAGTGGTGTGG |
| | (SEQ ID NO: 84) |
| GPX7_RUM | ACCCCAAATATTAACCACCTTAA |
| | (SEQ ID NO: 85) |
| GPX7_U_Probe | CTACTACACACAAACCACAACCCAC |
| | (SEQ ID NO: 86) |
| STD_GPX7_F | TGATAGTATTAGAAGGGATTGTAG |
| | (SEQ ID NO: 87) |
| STD_GPX7_R | CAAATCTAACCACATCCAAACTC |
| | (SEQ ID NO: 88) |
| STD_GPX7_Probe | AAATCACCTACCAATTCAACCATACCA |
| | (SEQ ID NO: 89) |
| HIN1_Ext_R3 | AAACTACAAAACAAAACCAC |
| | (SEQ ID NO: 90) |
| HIN1_Ext_F2 | GTTTGTTAAGAGGAAGTTTT |
| | (SEQ ID NO: 91) |
| HIN1_FM | TAGGGAAGGGGGTACGGGTTT |
| | (SEQ ID NO: 92) |
| HIN1_RM | CGCTCACGACCGTACCCTAA |
| | (SEQ ID NO: 93) |
| HIN1_M_Probe | ACTTCCTACTACGACCGACGAACC |
| | (SEQ ID NO: 94) |
| HIN1_FUM2 | AAGTTTTTGAGGT TTGGGTAGGGA |
| | (SEQ ID NO: 95) |
| HIN1_RUM2 | ACCAACCTCACCCACACTCCTA |
| | (SEQ ID NO: 96) |
| HIN1_U_Probe | CAACTTCCTACTACAACCAACAAACC |
| | (SEQ ID NO: 97) |
| STD_HIN1_F | ATAATGTTAGTAGATTGGAGGAGTT |
| | (SEQ ID NO: 98) |
| STD_HIN1_R | AACCCACATAACATTCCACTTATC |
| | (SEQ ID NO: 99) |
| STD_HIN1_Probe | AAAGAGTGGGAGGATGTTAGTGATAAGTG |
| | (SEQ ID NO: 100) |
| HIST1H3C_Ext_F2 | GTGTGTGTTTTTATTGTAAATGG |
| | (SEQ ID NO: 101) |
| HIST1H3C_Ext_R2 | ATAAAATTTCTTCACRCCACC |
| | (SEQ ID NO: 102) |
| HIST1H3C_FM2 | AATAGTTCGTAAGTTTATCGGCG |
| | (SEQ ID NO: 103) |
| HIST1H3C_RM2 | CTTCACGCCACCGATAACCGA |
| | (SEQ ID NO: 104) |
| HIST1H3C_M_ | TACTTACGCGAAACTTTACCGCCGA |
| Probe | (SEQ ID NO: 105) |
| HIST1H3C_FUM2 | GTAAATAGTTTGTAAGTTTATTGGTG |
| | (SEQ ID NO: 106) |
| HIST1H3C_RUM2 | TTTCTTCACACCACCAATAACCAA |
| | (SEQ ID NO: 107) |
| HIST1H3C_U_ | AACTACTTACACAAAACTTTACCACCAA |
| Probe | (SEQ ID NO: 108) |
| STD_HIST1H3C_F | GATTTAGAGTTGGATGTGTGGAT |
| | (SEQ ID NO: 109) |
| STD_HIST1H3C_R | ACCACCATACTAATAATCAAATCTA |
| | (SEQ ID NO: 110) |
| STD_HIST1H3C_ | AAATATCACTCATCACCAAATAAATCCAA |
| Probe | (SEQ ID NO: 111) |
| HOXB4_Ext_F | TTAGAGGYGAGAGAGTAGTT |
| | (SEQ ID NO: 112) |
| HOXB4_Ext_R | AAACTACTACTAACCRCCTC |
| | (SEQ ID NO: 113) |
| HOXB4_FM | CGGGATTTTGGGTTTTCGTCG |
| | (SEQ ID NO: 114) |
| HOXB4_RM | CGACGAATAACGACGCAAAAAC |
| | (SEQ ID NO: 115) |
| HOXB4_M_Probe | AACCGAACGATAACGAAAACGACGAA |
| | (SEQ ID NO: 116) |
| HOXB4_FUM3 | GTGGTGTGTATTGTGTAGTGTTA |
| | (SEQ ID NO: 117) |
| HOXB4_RUM2 | CAAACCAAACAATAACAAAAACAAC |
| | (SEQ ID NO: 118) |
| HOXB4_U2_Probe | CAAAATCCCAACAAACCACATAACACT |
| | (SEQ ID NO: 119) |
| STD_HOXB4_F | GTTAGTTTTGTAGTGTATTGAGTAT |
| | (SEQ ID NO: 120) |
| STD_HOXB4_R | CATCTTCCACAATAAACTTCCAATT |
| | (SEQ ID NO: 121) |
| STD_HOXB4_Probe | TAACTCCACCTATTCTACCTACCATTT |
| | (SEQ ID NO: 122) |
| MAL_Ext_F | GATTTATAGTTTTTAGTTTTGGA |
| | (SEQ ID NO: 123) |
| MAL_Ext_R | AAACCACTAAACAAAATACTAC |
| | (SEQ ID NO: 124) |
| MAL_FM | TTTCGCGGAGTTAGCGAGAG |
| | (SEQ ID NO: 125) |
| MAL_RM | AAACCATAACGACGTACTAACG |
| | (SEQ ID NO: 126) |
| MAL_M_Probe | AAAACGAAACGAACGCCGCTCAAAC |
| | (SEQ ID NO: 127) |
| MAL_FUM | GTTTTGTGGAGTTAGTGAGAGG |
| | (SEQ ID NO: 128) |
| MAL_RUM | AAACCATAACAACATACTAACATC |
| | (SEQ ID NO: 129) |
| MAL_U_Probe | CTTAAAACAAAACAAACACCACTCAAAC |
| | (SEQ ID NO: 130) |
| STD_MAL_F | GTGTGGGATGTGTTTAGTGATTT |
| | (SEQ ID NO: 131) |
| STD_MAL_R | CAATCCTACACAAACATCAACAT |
| | (SEQ ID NO: 132) |
| STD_MAL_Probe | GGTGATGTGTTGTATGTTGGTATGG |
| | (SEQ ID NO: 133) |
| RARB Ext F | GTAGGAGGGTTTATTTTTTGTT |
| | (SEQ ID NO: 134) |
| RARB Ext R(2) | TTACCATTTTCCAAACTTACTC |
| | (SEQ ID NO: 135) |
| RARB FM | AGAACGCGAGCGATTCGAGTAG |
| | (SEQ ID NO: 136) |
| RARB RM | TACAAAAAACCTTCCGAATACGTT |
| | (SEQ ID NO: 137) |
| RARB_U_Probe | AAATCCTACCCCAACAATACCCAAAC |
| | (SEQ ID NO: 138) |
| RARB FUM | TTGAGAATGTGAGTGATTTGAGTAG |
| | (SEQ ID NO: 139) |
| RARB RUM | TTACAAAAAACCTTCCAAATACATTC |
| | (SEQ ID NO: 140) |
| RARB_M_Probe | ATCCTACCCCGACGATACCCAAAC |
| | (SEQ ID NO: 141) |
| RASGRF2_Ext_F | GAGGGAGTTAGTTGGGTTAT |
| | (SEQ ID NO: 142) |
| RASGRF2_Ext_R | CCTCCAAAAAATACATACCC |
| | (SEQ ID NO: 143) |
| RASGRF2_FM | GTAAGAAGACGGTCGAGGCG |
| | (SEQ ID NO: 144) |
| RASGRF2_RM | ACAACTCTACTCGCCCTCGAA |
| | (SEQ ID NO: 145) |
| RASGRF2_M_Probe | AACGAACCACTTCTCGTACCAACGA |
| | (SEQ ID NO: 146) |
| RASGRF2_FUM | GAGTAAGAAGATGGTTGAGGTG |
| | (SEQ ID NO: 147) |
| RASGRF2_RUM | CAACAACTCTACTCACCCTCAA |
| | (SEQ ID NO: 148) |
| RASGRF2_U_Probe | AAACAAACCACTTCTCATACCAACAAC |
| | (SEQ ID NO: 149) |
| STD_RASGRF2_F | TGTATGAGTTTGTGGTGAATAATG |
| | (SEQ ID NO: 150) |
| STD_RASGRF2_R | AACTCACCATCAAACACTTTCCC |
| | (SEQ ID NO: 151) |
| STD_RASGRF2_ | TACAAACCCAACATCCTCTATCTATTC |
| Probe | (SEQ ID NO: 152) |
| RASSF1_Ext_F2 | GTTTTATAGTTTTTGTATTTAGG |
| | (SEQ ID NO: 153) |
| RASSF1_Ext_R2 | AACTCAATAAACTCAAACTCCC |
| | (SEQ ID NO: 154) |
| RASSF1_FM | GCGTTGAAGTCGGGGTTC |
| | (SEQ ID NO: 155) |
| RASSF1_RM | CCCGTACTTCGCTAACTTTAAACG |
| | (SEQ ID NO: 156) |
| RASSF1_M-Probe | ACAAACGCGAACCGAACGAAACCA |
| | (SEQ ID NO: 157) |
| RASSF1_RT-FUM | GGTGTTGAAGTTGGGGTTTG |
| | (SEQ ID NO: 158) |
| RASSF1_RT-RUM | CCCATACTTCACTAACTTTAAAC |
| | (SEQ ID NO: 159) |
| RASSF1_U_Probe | CTAACAAACACAAACCAAACAAAACCA |
| | (SEQ ID NO: 160) |
| STD_RASSF1_F2 | TTAGGGTAGATTGTGGATATTAG |
| | (SEQ ID NO: 161) |
| STD_RASSF1_R3 | ATACTAACAACTATCCAATACAAC |
| | (SEQ ID NO: 162) |
| STD_RASSF1_ | AGGTTGAAATTAGTATGTGTTATTTTGGTATGG |
| Probe 2 | (SEQ ID NO: 163) |
| TM6SF1_Ext_F | AGGAGATATYGTTGAGGGGA |
| | (SEQ ID NO: 164) |
| TM6SF1_Ext_R | TCACTCATACTAAACCRCCAA |
| | (SEQ ID NO: 165) |
| TM6SF1_FM | CGTTTAGCGGGATGCGGTGA |
| | (SEQ ID NO: 166) |
| TM6SF1_RM | ACACGAAAACCCCGATAACCG |
| | (SEQ ID NO: 167) |
| TM6SF1_M_Probe | AAACACTCATCGCAACCGCCGCG |
| | (SEQ ID NO: 168) |
| TM6SF1_FUM | TGTTTAGTGGGATGTGGTGAAG |
| | (SEQ ID NO: 169) |
| TM6SF1_RUM | ACACAAAAACCCCAATAACCACA |
| | (SEQ ID NO: 170) |
| TM6SF1_U_Probe | AAACACTCATCACAACCACCACACC |
| | (SEQ ID NO: 171) |
| STD_TM6SF1_F | TTAGATGTTGATTGGTTGTGTTTG |
| | (SEQ ID NO: 172) |
| STD_TM6SF1_R | ATCATCATAAAACTCAACAATCAATT |
| | (SEQ ID NO: 173) |
| STD_TM6SF1_ | CCAAACATCAAATCTTTAACTTTTACCAA |
| Probe | (SEQ ID NO: 174) |
| TMEFF2_Ext F | TTATGGTAGTAGTTTTTYGYGTT |
| | (SEQ ID NO: 175) |
| TMEFF2_Ext R | CCCACAACACCATAACTAATTC |
| | (SEQ ID NO: 176) |
| TMEFF2_FM | TTTCGTTTCGGGGTTGAGTTTAG |
| | (SEQ ID NO: 177) |
| TMEFF2_RM | ACGATAACAATAACACCCGACGA |
| | (SEQ ID NO: 178) |
| TMEFF2_M_Probe | CAAACCCGCGCATAATCTCGAAAATT |
| | (SEQ ID NO: 179) |
| TMEFF2_FUM | TTTTGTTTTGGGGTTGAGTTTAGTT |
| | (SEQ ID NO: 180) |
| TMEFF2_RUM | CAACAATAACAATAACACCCAACAA |
| | (SEQ ID NO: 181) |
| TMEFF2_U_Probe | CAAACCCACACATAATCTCAAAAATTTC |
| | (SEQ ID NO: 182) |
| STD_TMEFF2_F | ATTAGTGAAGGGTTGATTGAAGG |
| | (SEQ ID NO: 183) |
| STD_TMEFF2_R | CCAAATATATTAATATTCCCCTCAA |
| | (SEQ ID NO: 184) |
| STD_TMEFF2_ | ACCAACATACTATTCAACAACACACTTT |
| Probe | (SEQ ID NO: 185) |
| TWIST_Ext_F3 | GAGATGAGATATTATTTATTGTG |
| | (SEQ ID NO: 186) |
| TWIST_Ext_R4 | CCTCCCAAACCATTCAAAAAC |
| | (SEQ ID NO: 187) |
| TWIST_FM | GTTAGGGTTCGGGGGCGTTGTT |
| | (SEQ ID NO: 188) |
| TWIST_RM | CCGTCGCCTTCCTCCGACGAA |
| | (SEQ ID NO: 189) |
| TWIST_M_Probe | AAACGATTTCCTTCCCCGCCGAAA |
| | (SEQ ID NO: 190) |
| TWIST_FUM3a | TTAGGGTTTGGGGGTGTTGTTTGTATG |
| | (SEQ ID NO: 191) |
| TWIST_RUM5 | CCATCACCTTCCTCCAACAAAC |
| | (SEQ ID NO: 192) |
| TWIST_U_Probe | AAACAATTTCCTTCCCCACCAAAACA |
| | (SEQ ID NO: 193) |
| STD_TWIST_F | TTGTATTTATTGATTTGGTAAATGGG |
| | (SEQ ID NO: 194) |
| STD_TWIST_R | ACATCATTCATAAATATCTAATTACC |
| | (SEQ ID NO: 195) |
| STD_TWIST_ | ACACCACAAACATCAACATTTCATTCCC |
| Probe | (SEQ ID NO: 196) |
| ZNF671_EXT_F | TAGGTGGAGGTGTTGGGAAA |
| | (SEQ ID NO: 197) |
| ZNF671_EXT_R | CTATCCTAAAACACAAAAACTAC |
| | (SEQ ID NO: 198) |
| ZNF671_FM1 | GTGTTTCGAGACGCGTTTGATG |
| | (SEQ ID NO: 199) |
| ZNF671_RM1 | AACTACCGAAAACGACAAACGTC |
| | (SEQ ID NO: 200) |
| ZNF671_M_Probe | ATCGAAAACGCAAACACTTCCGTCC |
| | (SEQ ID NO: 201) |
| ZNF671_FUM1 | TTAGTGTTTTGAGATGTGTTTGATG |
| | (SEQ ID NO: 202) |
| ZNF671_RUM | ACAACTACCAAAAACAACAAACATC |
| | (SEQ ID NO: 203) |
| ZNF671_U_Probe | AATCAAAAACACAAACACTTCCATCCCT |
| | (SEQ ID NO: 204) |
| STD_ZNF671_F | TAGGAGATGTTGATTAAGGTAGAG |
| | (SEQ ID NO: 205) |
| STD_ZNF671_R | AACAAAATAATCATCCTTACACAAATT |
| | (SEQ ID NO: 206) |
| STD_ZNF671_ | ATATCATCTACTTTCTTATACACAACCTC |
| Probe | (SEQ ID NO: 207) |

It will be understood by those of ordinary skill in the art, that the detection of methylation of the CpG regions of the genes and the level of methylation detected in the samples from suspicious lesions of a subject is compared to the methylation levels of the CpG regions of the genes in normal tissue or benign lesions. When the level is elevated in the sample compared to normal control tissue or benign lesions, the lesion is diagnosed as having a high risk of cancer or malignancy. The particular gene panel of the inventive methods were specifically chosen to identify those genes which were very highly methylated when malignant or cancerous, and had little or no methylation when in normal or benign tissue. Moreover, when the level of methylation of the genes in the sample from the mammographically suspicious lesion is not significantly different that normal or benign tissue, there is a low probability or risk of cancer in the lesion sampled.

The inventive methods herein employ two-step quantitative multiplex-methylation specific PCR (QM-MSP). The present inventors invented this method, and these methods are disclosed in U.S. Pat. Nos. 8,062,849 and 9,416,404, both of which are hereby incorporated herein as if set forth in their entireties. The inventive methods can also employ the QM-MSP variant method, cMethDNA to measure hypermethylation in a sample. The present inventors invented this method and these methods are disclosed in U.S. Pat. No. 10,450,609, and U.S. patent application Ser. No. 16/601,269, both of which are hereby incorporated herein as if set forth in their entireties. The QM-MSP technique combines the sensitivity of multiplex PCR with the quantitative features of quantitative methylation-specific PCR (Q-MSP) in such a way that a panel of genes whose hypermethylation is associated with a type of carcinoma can be co-amplified from limiting amounts of DNA derived from tissue or samples sources of the subject being tested. The invention methods also provide quantitative definition of the extent of gene hypermethylation in normal appearing tissues on a gene-by-gene basis. Thus, the inventive methods can be used to more powerfully discriminate between normal or benign tissues and malignant tissues and to monitor or assess the course of cancer development in a subject.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

It will be understood by those of ordinary skill in the art that the methods of the present invention can be used to diagnose, prognosticate, and monitor treatment of any disease or biological state in which methylation of genes is correlative of such a disease or biological state in a subject. In some embodiments, the disease state is breast cancer. In some embodiments, the type of breast cancer can be invasive ductal carcinoma or ductal carcinoma in situ.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream. In preferred embodiments, the cancers include breast, colon, and lung cancer.

The cancer can be a metastatic cancer or a non-metastatic (e.g., localized) cancer, an invasive cancer or an in situ cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

As used herein, the term “triaging the subject into core breast biopsy of the suspicious lesion and pathological review” means that when the sample DNA has two or more markers methylated, the subject is then selected for FNA or biopsy of the suspect tissue lesion or node which undergoes pathological review. Upon a diagnosis of cancer or possible malignancy, the tissues are further analyzed for ER/PR/HER2 subtyping of the tumor by immunohistochemistry and FISH analysis is necessary to deliver appropriate care.

Appropriate care in terms of breast cancer can constitute standard of care for treatment of breast cancer including, for example, surgery, surgery with post-operative radiation therapy, post-operative systemic therapy or chemotherapy depending on whether he tumor is hormone receptor negative or positive, the tumor is HER2/neu negative or positive, the tumor is hormone receptor negative and HER2/neu negative (triple negative), and the size of the tumor. Chemotherapy for breast cancer can include In premenopausal women with hormone receptor positive tumors, no more treatment may be needed or postoperative therapy may include: tamoxifen therapy with or without chemotherapy; tamoxifen therapy and treatment to stop or lessen how much estrogen is made by the ovaries; drug therapy, surgery to remove the ovaries, or radiation therapy to the ovaries may be used; aromatase inhibitor therapy and treatment to stop or lessen how much estrogen is made by the ovaries; and drug therapy, surgery to remove the ovaries, or radiation therapy to the ovaries may be used.

In postmenopausal women with hormone receptor positive tumors, no more treatment may be needed or postoperative therapy may include: aromatase inhibitor therapy with or without chemotherapy; tamoxifen followed by aromatase inhibitor therapy, with or without chemotherapy.

In women with hormone receptor negative tumors, no more treatment may be needed or postoperative therapy may include chemotherapy.

In women with small, HER2/neu positive tumors, and no cancer in the lymph nodes, no more treatment may be needed. If there is cancer in the lymph nodes, or the tumor is large, postoperative therapy may include: chemotherapy and targeted therapy (trastuzumab); hormone therapy, such as tamoxifen or aromatase inhibitor therapy, for tumors that are also hormone receptor positive.

Drugs useful in the treatment of breast cancer include, but are not limited to: Abemaciclib; Abraxane (Paclitaxel Albumin-stabilized Nanoparticle Formulation); Ado-Trastuzumab Emtansine; Afinitor (Everolimus); Anastrozole; Aredia (Pamidronate Disodium); Arimidex (Anastrozole); Aromasin (Exemestane); Capecitabine; Cyclophosphamide; Docetaxel; Doxorubicin Hydrochloride; Ellence (Epirubicin Hydrochloride); Epirubicin Hydrochloride; Eribulin Mesylate; Everolimus; Exemestane; 5-FU (Fluorouracil Injection); Fareston (Toremifene); Faslodex (Fulvestrant); Femara (Letrozole); Fluorouracil Injection; Fulvestrant; Gemcitabine Hydrochloride; Gemzar (Gemcitabine Hydrochloride); Goserelin Acetate; Halaven (Eribulin Mesylate); Herceptin Hylecta (Trastuzumab and Hyaluronidase-oysk); Herceptin (Trastuzumab); Ibrance (Palbociclib); Ixabepilone; Ixempra (Ixabepilone); Kadcyla (Ado-Trastuzumab Emtansine); Kisqali (Ribociclib); Lapatinib Ditosylate; Letrozole; Lynparza (Olaparib); Megestrol Acetate; Methotrexate; Neratinib Maleate; Nerlynx (Neratinib Maleate); Olaparib; Paclitaxel; Paclitaxel Albumin-stabilized Nanoparticle Formulation; Palbociclib; Pamidronate Disodium; Perjeta (Pertuzumab); Pertuzumab; Ribociclib; Talazoparib Tosylate; Talzenna (Talazoparib Tosylate); Tamoxifen Citrate; Taxol (Paclitaxel); Taxotere (Docetaxel); Thiotepa; Toremifene; Trastuzumab; Trastuzumab and Hyaluronidase-oysk; Trexall (Methotrexate); Tykerb (Lapatinib Ditosylate); Verzenio (Abemaciclib); Vinblastine Sulfate; Xeloda (Capecitabine); Zoladex (Goserelin Acetate); and combinations thereof.

As used herein, the term “triaging the subject into colon lesion biopsy of the suspicious lesion and pathological review” means that when the sample DNA has two or more markers methylated, the subject is then selected for biopsy of the suspect tissue lesion or node which undergoes pathological review. Upon a diagnosis of cancer or possible malignancy, the tissues are further analyzed for staging. There are seven different types of treatments for subject diagnosed with colon cancer including: Surgery; Radiofrequency; Ablation; Cryosurgery; Chemotherapy; Radiation therapy; Targeted therapy; and Immunotherapy. Drugs approved for use in chemotherapy include: Avastin (Bevacizumab) Bevacizumab; Camptosar (Irinotecan Hydrochloride); Capecitabine; Cetuximab; Cyramza (Ramucirumab); Eloxatin (Oxaliplatin); Erbitux (Cetuximab); 5-FU (Fluorouracil Injection); Fluorouracil Injection; Fusilev (Leucovorin Calcium); Ipilimumab; Irinotecan Hydrochloride; Keytruda (Pembrolizumab); Leucovorin Calcium; Lonsurf (Trifluridine and Tipiracil Hydrochloride); Nivolumab; Opdivo (Nivolumab); Oxaliplatin; Panitumumab; Pembrolizumab; Ramucirumab; Regorafenib; Stivarga (Regorafenib); Trifluridine and Tipiracil Hydrochloride; Vectibix (Panitumumab); Xeloda (Capecitabine); Yervoy (Ipilimumab); Zaltrap (Ziv-Aflibercept); Ziv-Aflibercept and combinations thereof.

As used herein, the term “triaging the subject into lung lesion biopsy of the suspicious lesion and pathological review” means that when the sample DNA has two or more markers methylated, the subject is then selected for biopsy of the suspect tissue lesion or node which undergoes pathological review. Upon a diagnosis of cancer or possible malignancy, the tissues are further analyzed for staging. Typical treatments include Surgery; Surgery and radiation therapy; Radiation therapy alone; and Chemotherapy combined with radiation therapy and/or surgery; Combination chemotherapy; Combination chemotherapy and targeted therapy with a monoclonal antibody, such as bevacizumab, cetuximab, or necitumumab; Combination chemotherapy followed by more chemotherapy as maintenance therapy to help keep cancer from progressing; Targeted therapy with an epidermal growth factor receptor (EGFR) tyrosine kinase inhibitor, such as osimertinib, gefitinib, erlotinib, or afatinib; Targeted therapy with an anaplastic lymphoma kinase (ALK) inhibitor, such as alectinib, crizotinib or ceritinib; Targeted therapy with a BRAF or MEK inhibitor, such as dabrafenib or trametinib; and Immunotherapy with an immune checkpoint inhibitor, such as pembrolizumab, with or without chemotherapy. Examples of drugs used in the treatment of non-small cell lung cancer include, but are not limited to: Abraxane (Paclitaxel Albumin-stabilized Nanoparticle Formulation) Afatinib Dimaleate; Afinitor (Everolimus); Alecensa (Alectinib); Alectinib; Alimta (Pemetrexed Disodium); Alunbrig (Brigatinib); Atezolizumab; Avastin (Bevacizumab); Bevacizumab; Brigatinib; Carboplatin; Ceritinib; Crizotinib; Cyramza (Ramucirumab); Dabrafenib; Dacomitinib; Docetaxel; Durvalumab; Erlotinib Hydrochloride; Everolimus; Gefitinib; Gilotrif (Afatinib Dimaleate); Gemcitabine Hydrochloride; Gemzar (Gemcitabine Hydrochloride); Imfinzi (Durvalumab); Iressa (Gefitinib); Keytruda (Pembrolizumab); Lorbrena (Lorlatinib); Lorlatinib; Mechlorethamine Hydrochloride; Mekinist (Trametinib); Methotrexate; Mustargen (Mechlorethamine Hydrochloride); Navelbine (Vinorelbine Tartrate); Necitumumab; Nivolumab; Opdivo (Nivolumab); Osimertinib; Paclitaxel; Paclitaxel Albumin-stabilized Nanoparticle Formulation; Paraplat (Carboplatin); Paraplatin (Carboplatin); Pembrolizumab; Pemetrexed Disodium; Portrazza (Necitumumab); Ramucirumab; Tafinlar (Dabrafenib); Tagrisso (Osimertinib); Tarceva (Erlotinib Hydrochloride); Taxol (Paclitaxel); Taxotere (Docetaxel); Tecentriq (Atezolizumab); Trametinib; Trexall (Methotrexate); Vizimpro (Dacomitinib); Vinorelbine Tartrate; Xalkori (Crizotinib); and Zykadia (Ceritinib).

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, staging, screening, or other patient management, including treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules.

As used herein, the term “selective hybridization” or “selectively hybridize” refers to hybridization under moderately stringent or highly stringent conditions such that a nucleotide sequence associates with a selected nucleotide sequence, but not with unrelated nucleotide sequences. Generally, an oligonucleotide useful as a probe or primer that selectively hybridizes to a selected nucleotide sequence is at least about 15 nucleotides in length, usually at least about 18 nucleotides, and particularly about 20 nucleotides in length or more in length. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual” (Cold Spring Harbor Laboratory Press 1989)).

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

As used herein, the term “optically detectable DNA probe” means an oligonucleotide probe that can act as a molecular beacon or an oligonucleotide probe comprising a fluorescent moiety or other detectable label, with or without a quencher moiety.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

Use of Quantitative Multiplex Methylation Specific PCR

QM-MSP is a highly sensitive, specific and quantitative methylation assay. It combines the principles of conventional gel-based MSP, quantitative real time MSP, and multiplexed gel-based MSP into one format developed to enable quantification of methylated gene panels in clinical samples with limited DNA quantities available. It has an analytical sensitivity of 1 methylated copies in 100,000 unmethylated copies, which is nearly 10 fold higher than QMSP, and 100 fold higher than gel based MSP techniques.

QM-MSP is a two-step PCR approach for a multiplexed analysis of a panel of up to 14 genes in clinical samples with minimal quantities of DNA. In the first step, the External PCR reaction, for up to 14 genes tested, one pair of gene-specific primers (forward and reverse) amplifies the methylated and unmethylated copies of the same gene simultaneously and in multiplex, in one PCR reaction. This is a methylation-independent amplification step, to increase the number of DNA segments. In the second step, methylated (M) and unmethylated (U) primers are used which selectively amplify methylated and un-methylated DNA, and the amplicons are subsequently quantified with a standard curve using real-time PCR and two independent fluorophores to detect methylated/unmethylated DNA of each gene in the same well. Methylation is reported on a continuous scale.

The assay is easily performed on fresh or fixed cytological samples including ductal lavage/ductoscopy fluids and cells, nipple fluids, and fine needle aspirates as well as tissues and core biopsies.

Primer and Probe Design for QM-MSP.

In practice of the methods of the present invention, quantitative real-time PCR methodology is adapted to perform quantitative methylation-specific PCR (QM-MSP) by utilizing the external primers pairs in round one (multiplex) PCR and internal primer pairs in round two (real time MSP) PCR. Thus, each set of genes has one pair of external primers and two sets of three internal primers/probe (internal sets are specific for unmethylated or methylated DNA). The external primer pairs can co-amplify a cocktail of genes, each pair selectively hybridizing to a member of the panel of genes being investigated using the invention method. Primer pairs are designed to exclude CG dinucleotides, thereby rendering DNA amplification independent of the methylation status of the promoter DNA sequence. Therefore, methylated and unmethylated DNA sequences internal to the binding sites of the external primers are co-amplified for any given gene by a single set of external primers specific for that gene. The external primer pair for a gene being investigated is complementary to the sequences flanking the CpG regions that is to be queried in the second round of QM-MSP. For example, the sequences of external primers set forth in Table 1 above are used for multiplex PCR (first round PCR) of genes associated with primary breast cancer (Fackler M. J. et al, Int. J Cancer (2003) 107:970-975; Fackler M. J. et al. Cancer Res (2004) 64:4442-4452).

Internal PCR primers used for quantitative real-time PCR of methylated and unmethylated DNA sequences (round two QM-MSP) are designed to selectively hybridize to the first amplicon produced by the first round of PCR for one or more members of the panel of DNA sequences being investigated using the invention method and to detect the methylation status, i.e., whether methylated (M) or unmethylated (U), of the CpG regions in the first amplicons to which they bind. Thus for each member of the starting panel of promoter DNA sequences used in an invention assay, separate QM-MSP reactions are conducted to amplify the first amplicon produced in the first round of PCR using the respective methylation-specific primer pair and using the respective unmethylated-specific primer pair.

In round two of QM-MSP a single gene or a cocktail of two or more genes can be co-amplified using distinguishable fluorescence labeled probes. The probes used in the round two QM-MSP of the invention method are designed to selectively hybridize to a segment of the first amplicon lying between the binding sites of the respective methylation-status specific internal PCR primer pair. Polynucleotide probes suitable for use in real-time PCR include, but are not limited to, a bi-labeled oligonucleotide probe, such as a molecular beacon or a TaqMan™ probe, which include a fluorescent moiety and a quencher moiety. In a molecular beacon the fluorescence is generated due to hybridization of the probe, which displaces the quencher moiety from proximity of the fluorescent moiety due to disruption of a stem-loop structure of the bi-labeled oligonucleotide. Molecular beacons, such as Amplifluor™ or TriStar™ reagents and methods are commercially available (Stratagene; Intergen). In a TaqMan™ probe, the fluorescence is progressively generated due to progressive degradation of the probe by Taq DNA polymerase during rounds of amplification, which displaces the quencher moiety from the fluorescent moiety. Once amplification occurs, the probe is degraded by the 5′-3′ exonuclease activity of the Taq DNA polymerase, and the fluorescence can be detected, for example by means of a laser integrated in the sequence detector. The fluorescence intensity, therefore, is proportional to the amount of amplification product produced.

In one embodiment, fluorescence from the probe is detected and measured during a linear amplification reaction and, therefore, can be used to detect generation of the linear amplification product.

Amplicons in the 80-150 base pair range are generally favored because they promote high-efficiency assays that work the first time. In addition, high efficiency assays enable quantitation to be performed using the absolute quantitation method. Quantitation of the copy number of unmethylated (U) and methylated (M) DNA amplicons for a specific gene eliminates the need to use actin as an estimate of DNA input, since U+M is taken to approximate the total number of copies of DNA amplicons for any given gene in the first amplicon product (derived from round one, multiplex PCR of QM-MSP).

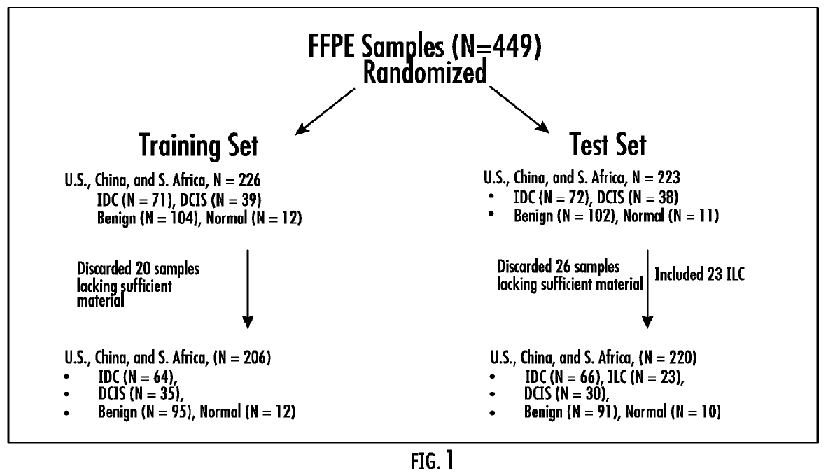

The TaqMan™ probe used in the Example herein contains both a fluorescent reporter dye at the 5′ end, such as 6-carboxyfluorescein (6-FAM: emission λmax=518 nm) and a quencher dye at the 3′ end, such as 6-carboxytetramethylrhodamine, (TAMRA; emission λmax=582 nm). The quencher can quench the reporter fluorescence when the two dyes are close to each other, which happens in an intact probe. Other reporter dyes include but are not limited to VIC™ and TET™ and these can be used in conjunction with 6-FAM to co-amplify genes by quantitative real time PCR. For instance, in round two QM-MSP, unmethylated (using a 6-FAM/TAMRA probe) and unmethylated RARE (using a VIC/TAMRA probe) either can be co-amplified (FIG. 1) or can be assayed as single genes.

Thermal Cycling Parameters

The round two QM-MSP reactions are designed to be run as single gene reactions or in multiplex using automated equipment, as are other types of real time PCR. Thermal cycling parameters useful for performing real time PCR are well known in the art and are illustrated in the Examples herein. In certain embodiments, quantitative assays can be run using the same universal thermal cycling parameters for each assay. This eliminates any optimization of the thermal cycling parameters and means that multiple assays can be run on the same plate without sacrificing performance. This benefit allows combining two or more assays into a multiplex assay system, in which the option to run the assays under different thermal cycling parameters is not available.

Using the QM-MSP approach, it is possible to compile gene panels that are designed to address specific questions, or to provide intermediate markers or endpoints for clinical protocols. For example, when using de-methylating agents, a panel can be designed to query pathway-specific genes for their use as intermediate markers in specific trials of chemopreventive agents (Fackler M. J. et al. J Mammary Gland Biol Neoplasia (2003) 8:75-89).

In some embodiments, the real-time PCR amplification in an invention two-step assay is, in fact, a group of real-time PCR reactions, which may be conducted together or using two separate aliquots of the first amplification product, for each of the first amplicons (i.e., for each DNA sequence in the panel that were selected for the assay). In these embodiments, determination of the methylation status of a DNA sequence, such as one containing a CpG island, employs both a methylation-determining and an unmethylation determining internal primer pair for each amplicon contained in the first amplification product, one to determine if the gene is unmethylated and one to determine if the gene is methylated. The real time PCR reactions in the second amplification step of the methods can conveniently be conducted sequentially or simultaneously in multiplex. Separate, usually dilute, aliquots of the first amplification reaction may be used for each of the two methylation status determining reactions. For example, the reactions can conveniently be performed in the wells of a 96 or 384 microtiter plate. For convenience, the methylated and unmethylated status determining second reactions for a target gene may be conducted in adjacent wells of a microtiter plate for high throughput screening. Alternatively, several genes, for example 2 to 5 genes, may be simultaneously amplified in a single real time PCR reaction if the probes used for each first amplicon are distinguishably labeled.

For example, two to ten, or more distinguishable fluorescence signals from the second amplification product(s) may be accumulated to determine the cumulative incidence or level of methylation of the DNA sequences, especially of CpG regions therein, in the several genes included in the assay. These cumulative results are compared with the cumulative results similarly obtained by conducting the two-step QM-MSP assay on comparable DNA sequences (e.g., promoter DNA sequences) obtained from comparable normal tissue of the same type or types as used in the assay.

Any of the known methods for conducting cumulative or quantitative “real time PCR” may be used in the second amplification step so long as the first amplicons in the first amplification product are contacted with one or more members of a set of polynucleotide probes that are labeled with distinguishable optically detectable labels, one or more members of the set being designed to selectively hybridize to one or more of the DNA sequences being tested, while the set cumulatively binds to the various DNA segments being tested contained in the first amplicons of the first amplification product. In addition, the first amplicons may also be contacted with such a set of probes and one or more members of a set of DNA sequence-specific methylation status-dependent inner primer pairs, wherein the set of inner primer pairs collectively bind to the various first amplicons in the first amplification product. In round two QM-MSP, additional genes can be co-amplified provided that each gene primer set incorporates a different color fluorescent probe.

For example, when using the Applied Biosystem's 7500 Real-time PCR System, sample values are extrapolated from the standard curve for target and reference DNAs. This is called absolute quantitation.

QM-MSP: Percent Methylation (% M)=[copies Methylated TARGET gene/copies Methylated TARGET gene+copies un-methylated TARGET gene) copies] [100]; CMI=the sum of all % M values within the panel.

The phrase “a comparable normal DNA sample” as used herein means that the plurality of genomic DNA sequences that is being tested for methylation status, such as in a mammal, is matched with a panel of genomic DNA sequences of the same genes from a “normal” organism of the same species, family, and the like, for comparison purposes. For example, a substantial cumulative increase or decrease in the methylation level in the test sample as compared with the normal/benign sample (e.g., the cumulative incidence of the tumor marker in a test DNA panel compared with that cumulatively found in comparable apparently normal sample) is a reliable indicator of the presence of the condition being assayed.

Use of cMethDNA Methylation Specific PCR Methods

The cMethDNA methods combine the principles of QM-MSP with addition of STDgene standards which are engineered specifically for the TARGETgene of interest. It is known that QM-MSP sensitivity is 1:100,000 and this combination of procedures (cMethDNA) can detect as few as 50 methylated copies of DNA. This outcome compares favorably to Q-MSP with a sensitively of 1:10,000 and conventional MSP with a sensitivity of 1:1000. In addition, reactions are specific since no cross-reactivity was observed between TARGETgene and STDgene primers even in mixtures consisting of more than 10⁵-fold excess of one or the other DNA.

Primer and Probe Design for cMethDNA. In practice, the QM-MSP methodology is adapted to perform as cMethDNA by utilizing a single set of external primer pairs which hybridize to a single target gene of interest (TARGETgene) as well as to the respective STDgene for the TARGETgene in round one (pre-amplification) PCR in a manner independent of the methylation status of the TARGETgene, and in round two PCR (real time MSP) a methylation status-specific set of internal primers/probe hybridizing to the TARGETgene, as well as a TARGETgene standard-specific (i.e. STDgene) set of internal primers/probe hybridizing to the STDgene DNA. Thus each TARGETgene of interest has one pair of external primers and two sets of internal primer/probes, each internal set being designed specifically to detect and quantify the TARGETgene region of interest or the matched STDgene for each TARGETgene of interest.

A plurality of gene-specific external primer pairs may be used to co-amplify a plurality of TARGETgenes in what is termed multiplex PCR. Multiple external primer pairs used in the present inventive methods in one reaction can co-amplify a cocktail of TARGETgenes and their respective STDgenes, each pair selectively hybridizing to a member of the panel of TARGETgenes being investigated and their respective STDgenes using the inventive method. External primer pairs are designed to render DNA amplification independent of the methylation status of the DNA sequence. Therefore, methylated TARGETgene and STDgene DNA sequences internal to the binding sites of the external primers are co-amplified for any given gene by a single set of external primers specific for that TARGETgene (and later quantified by real-time PCR using internal primers). For example, the sequences of the primers set forth in Table 1, and in the accompanying sequence listing, are used with the methods of the present invention are TARGETgenes associated with primary breast cancer.

Internal PCR primers used for quantitative real-time PCR of methylated TARGETgene DNA sequences of interest and for the respective STDgene for the targeted endogenous gene(s) of interest (round two cMethDNA) are designed to selectively hybridize to the amplicon(s) produced by the first round of PCR for one or more members of the panel of TARGETgene DNA sequences being investigated, using the inventive methods to detect the methylation status, i.e., whether methylated (M) CpG residues are present within the amplified region of the CpG island(s). Thus for each member of the starting panel of DNA sequences used in an invention assay, separate round two cMethDNA reactions are conducted to amplify the amplicons produced in the first round of PCR using the respective methylation-specific internal primer pair and using the respective STDgene-specific internal primer pair. Round two of the cMethDNA methodology quantifies amplicons from regions of the TARGETgene and STDgene synthesized in round one. During round two real-time PCR the TARGETgene and respective STDgene can be assayed separately in individual wells or together in one well if both internal primer/probe sets are present in the same well and two differentiable detectable labels, such as distinctly different fluorescent labels and quenchers are used for the respective probes (e.g. 6FAM/TAMRA and VIC/TAMRA).

As used herein, the term “STDgene” means an oligonucleotide which is recombinantly inserted into a carrier DNA sequence to match the targeted endogenous gene (TARGETgene) of interest. Nucleotides at the 5′ and 3′ ends of the STDgene (approximately 20-22 bp hybridizing to forward and reverse external primers) are the same in sequence as the endogenous genomic DNA of interest (TARGETgene); and the number of intervening bases between the 5′ and 3′ regions in the STDgene oligonucleotide is essentially the same as the region of interest within the TARGETgene. To prevent cross-hybridization with human DNA, in an embodiment, the intervening bases between 5′ and 3′ ends consist of lambda phage DNA in the standard. To prevent cross-hybridization between standards (e.g. during the first round multiplex reaction), in an embodiment, each STDgene type has a unique lambda phage DNA sequence.

In accordance with one or more alternate embodiments, the intervening bases comprise any irrelevant DNA lacking homology to the region of the gene(s) of interest. This combination of features allows for co-amplification of the TARGETgene and STDgene in the multiplex reaction using a single set of external forward and reverse primers and later quantitation of these amplicons using specific primer/probes by real-time PCR in round two.

In round one multiplex PCR; there is a direct relationship between the number of copies of TARGETgene and STDgene DNAs amplified because each external forward and reverse primer has an equal chance of hybridizing to the target as it has to the STDgene (cloned to have 5′ and 3′ ends identical to the TARGETgene), providing the TARGETgene is present. Thus, for each TARGETgene the cMethDNA set includes: 1) external primers, forward and reverse, 2) TARGETgene methylation status-specific internal primers, forward and reverse, 3) STDgene-specific internal primers, forward and reverse, 4) probes to match #2 and #3, individually in distinct colors (e.g., 6FAM/TAMRA or VIC/TAMRA, or other combinations).

In round two of cMethDNA, when a single gene or standard is amplified, the event is detected with a distinguishable fluorescence labeled probe, and the intensity of signal doubles with each round of PCR (at 100% efficiency). The probes used in round two cMethDNA of the inventive methods are designed to selectively hybridize to the segment of the amplicon lying between the binding sites of the respective internal PCR primer pair. Polynucleotide probes suitable for use in real-time PCR include, but are not limited to, a bi-labeled oligonucleotide probe, such as a molecular beacon or a TaqMan™ probe, which include a fluorescent moiety and a quencher moiety. In a molecular beacon the fluorescence is generated due to hybridization of the probe, which displaces the quencher moiety from proximity of the fluorescent moiety due to disruption of a stem-loop structure of the bi-labeled oligonucleotide. Molecular beacons, such as Amplifluor™ or TriStar™ reagents and methods are commercially available (Stratagene; Intergen). In a TaqMan™ probe, the fluorescence is progressively generated due to progressive degradation of the probes by Taq DNA polymerase during rounds of amplification, which displaces the quencher moiety from the fluorescent moiety. Once amplification occurs, the probe is degraded by the 5′-3′ exonuclease activity of the Taq DNA polymerase, and the fluorescence can be detected, for example by means of a laser integrated in the sequence detector. The fluorescence intensity, therefore, is proportional to the amount of amplification product produced. Examples of fluorescent dyes which can be used with the inventive methods include 6-FAM, JOE, TET, Cal FLUOR Gold, Cal FLUOR Orange, Cal FLUOR Red, HEX, TAMRA, Cy3, Cy5, Cy5.5, Quasar 570, Quasar 670, ROX, and Texas Red. Examples of quenchers which can be used with the inventive methods include BHQ-1, BHQ-2, BHQ-3, and TAMRA.

In an embodiment, fluorescence from the probes are detected and measured during a linear amplification reaction and, therefore, can be used to detect the copy number of the amplification product.

In some embodiments, amplicons in the 75-150 base pair range are generally favored for the internal PCR assay because they promote high-efficiency assays which enable absolute quantitation to be performed. Quantitation of the copy number of STDgene and methylated TARGETgene amplicons for a specific gene is an improvement over the need to use as a reference unmethylated (U) DNA of the same gene or actin to estimate of DNA input, as described in the Background. Each respective STDgene has been designed with similar structural features (same amplicon length, and homologous bases at the 5′ and 3′ ends) as the region of interest in the specific TARGETgene (derived from round one, multiplex PCR of cMethDNA).

Whenever possible, primers and probes for the target gene region of interest can be selected in a region with a G/C content of 20-80%. Regions with G/C content in excess of this may not denature well during thermal cycling, leading to a less efficient reaction. In addition, G/C-rich sequences are susceptible to non-specific interactions that may reduce reaction efficiency. For this reason, primer and probe sequences containing runs of four or more G bases are generally avoided. A/T-rich sequences require longer primer and probe sequences in order to obtain the recommended melting temperatures (TMs). This is rarely a problem for quantitative assays; however, TaqMan™ probes approaching 40 base pairs can exhibit less efficient quenching and produce lower synthesis yields.

The TaqMan™ probe used in the Example herein contains both a fluorescent reporter dye at the 5′ end, such as 6-carboxyfluorescein (6-FAM: emission λmax=518 nm) and a quencher dye at the 3′ end, such as 6-carboxytetramethylrhodamine, (TAMRA; emission λmax=582 nm). The quencher can quench the reporter fluorescence when the two dyes are close to each other, which happens in an intact probe. Other reporter dyes include but are not limited to VIC™ and TET™ and these can be used in conjunction with 6-FAM to co-amplify genes by quantitative real time PCR. Other reporter constructs with or without a quencher moiety may also be used.

The inventive methods can be used to assess the methylation status of multiple genes, using very small quantities of DNA. A cumulative score of hypermethylation among multiple genes better distinguishes normal or benign from malignant tumors in bodily fluid samples as compared to the value of individual gene methylation markers. Using the cMethDNA methods of the present invention, it is possible to objectively define the range of normal/abnormal DNA sequence hypermethylation in a manner that is translatable to a larger clinical setting. The inventive methods may also be used to examine cumulative hypermethylation in benign conditions and as a predictor of conditions, such as various cancers and their metastases that are associated with DNA hypermethylation.

In real-time PCR, as used in the inventive methods, one or more aliquots (usually dilute) of the first amplification product is amplified with at least a first primer of an internal amplification primer pair, which can selectively hybridize to one or more amplicon in the first amplification product, under conditions that, in the presence of a second primer of the internal amplification primer pair, and a fluorescent probe allows the generation of a second amplification product. Simultaneously, at least one or more internal primer pairs specific for the STDgene for the TARGETgene(s) of interest also can selectively hybridize the STDgene in the presence of a second fluorescent probe specific for the STDgene. Detection of fluorescence from the second amplification product(s) provides a means for real-time detection of the generation of a second amplification product and for calculation of the amount of methylated TARGETgene and associated STDgene.

Two sequential PCR reactions are cMethDNA PCR reaction #2 (Real-time Reaction): For the real-time PCR reaction, the amplicons from Reaction #1 were quantified using two-color real-time PCR, according to the absolute quantitation method. Each TARGETgene and its paired reference STDgene were assayed independently, but in the same well. Controls were used in each reaction to insure specific hybridization to target or reference, as well as the absence of contamination. For cMethDNA the Methylation Index (MI) was calculated for each gene using the formula:

$$MI gene = \frac{M gene copies}{Total ( M gene + STD gene ) copies} ( 100 ) .$$

The cumulative methylation index (CMI) was calculated as the sum of MI for all genes performed.

cMethDNA PCR reaction #1 (Multiplex Reaction): In the multiplex reaction, DNA was amplified using “external” primers specific to sequences flanking the methylated region of interest. In this example, up to 12 genes per 50 μl reaction were co-amplified from ≥150 pg DNA template (50 copies of genomic DNA). The DNA yielded from 300 μl serum containing 50 copies of standard DNA was processed after bisulfite conversion.

General Conditions

It should be recognized that an amplification “primer pair” as the term is used herein requires what are commonly referred to as a forward primer and a reverse primer, which are selected using methods that are well known and routine and as described herein such that an amplification product can be generated therefrom.

As used herein, the phrase “conditions that allow generation of an amplification product” or of “conditions that allow generation of a linear amplification product” means that a sample in which the amplification reaction is being performed contains the necessary components for the amplification reaction to occur. Examples of such conditions are provided herein and include, for example, appropriate buffer capacity and pH, salt concentration, metal ion concentration if necessary for the particular polymerase, appropriate temperatures that allow for selective hybridization of the primer or primer pair to the template nucleic acid molecule, as well as appropriate cycling of temperatures that permit polymerase activity and melting of a primer or primer extension or amplification product from the template or, where relevant, from forming a secondary structure such as a stem-loop structure. Such conditions and methods for selecting such conditions are routine and well known in the art (see, for example, Innis et al., “PCR Strategies” (Academic Press 1995); Ausubel et al., “Short Protocols in Molecular Biology” 4th Edition (John Wiley and Sons, 1999); “A novel method for real time quantitative RT-PCR” Gibson U. E. et al. Genome Res (1996) 6:995-1001; “Real time quantitative PCR” Heid C. A. et al. Genome Res (1996) 6:986-994).

As used herein, the term “selective hybridization” or “selectively hybridize” refers to hybridization under moderately stringent or highly stringent conditions such that a nucleotide sequence associates with a selected nucleotide sequence, but not with unrelated nucleotide sequences. Generally, an oligonucleotide useful as a probe or primer that selectively hybridizes to a selected nucleotide sequence is at least about 15 nucleotides in length, usually at least about 18 nucleotides, and particularly about 21 nucleotides in length or more in length. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual” (Cold Spring Harbor Laboratory Press 1989)).

In various embodiments, methylation-specific PCR can be used to evaluate methylation status of the target DNA. MSP utilized primer and/or probe sets designed to be “methylated-specific” by including sequences complementing only unconverted 5-methylcytosines, or, on the converse, “unmethylated-specific”, complementing thymine's converted from unmethylated cytosines. Methylation is then determined by the ability of the specific primer to achieve amplification. This method is particularly effective for interrogating CpG islands in regions of high methylation density, because increased numbers of unconverted methylcytosines within the target to be amplified increase the specificity of the PCR. In certain embodiments placing the CpG pair at the 3′-end of the primer also improves the specificity.

In certain embodiments, methylation is evaluated using a MethyLight method. The MethyLight method is based on MSP, but provides a quantitative analysis using quantitative PCR (see, e.g., Eades et al. (2000) Nucleic Acids Res., 28(8): E32. doi:10.1093/nar/28.8.e32). Methylated-specific primers are used, and a methylated-specific fluorescence reporter probe is used that anneals to the amplified region. In alternative fashion, the primers or probe can be designed without methylation specificity if discrimination is needed between the CpG pairs within the involved sequences. Quantitation can be made in reference to a methylated reference DNA. One modification to this protocol to increase the specificity of the PCR for successfully bisulphite-converted DNA (ConLight-MSP) uses an additional probe to bisulphite-unconverted DNA to quantify this non-specific amplification (see, e.g., Rand et al. (2002)Methods 27(2): 114-120).

In various embodiments, the MethyLight methods utilize TAQMAN® technology, which is based on the cleavage of a dual-labeled fluorogenic hybridization probe by the 5′ nuclease activity of Taq-polymerase during PCR amplification (Eads et al. (1999) Cancer Res., 59: 2302-2306; Livak et al. (1995) PCR Meth. Appl., 4: 357-362; Lee et al. (1993) Nucleic Acids Res., 21: 3761-3766; Fink et al. (1998) Nat. Med., 4: 1329-1333). The use of three different oligonucleotides in the TAQMAN® technology (forward and reverse PCR primers and the fluorogenic hybridization probe) offers the opportunity for several sequence detection strategies.

In various embodiments, the methods described herein can involve nested PCR reactions and the reagents (e.g., primers and probes) for such nested PCR reactions. For example, in certain embodiments, methylation is detected for one, two, three, four, five, or six genes (gene promoters). Since bisulfite conversion of a DNA changes cytosine resides to uracil, but leave 5-methyl cytosine residues unaffected, the forward and reverse strands of converted (bisulfite-converted) DNA are no longer complementary. Accordingly, it is possible to interrogate the forward and reverse strands independently (e.g., in a multiplex PCR reaction) to provide additional specificity and sensitivity to methylation detection. In such instances, assaying of a single target can involve a two-plex multiplex assay, while assaying of two, three, four, five, or six target genes can involve four-plex, six-plex, 8-plex, 10-plex, or 12-plex multiplex assays. In certain embodiments the assays can be divided into two multiplex reactions, e.g., to independently assay forward and reverse strands. However, it will be recognized that when split into multiple multiplex assays, the grouping of assays need not be by forward or reverse, but can simply include primer/probe sets that are most compatible for particular PCR reaction conditions.

As indicated above, numerous cancers can be identified, and/or staged and/or a prognosis therefor determined by the detection/characterization of the methylation state on the forward and/or reverse strand of gene promoters whose methylation (or lack thereof) is associated with a cancer. It will be recognized that methylation (forward strand and/or reverse strand) of one or more of the genes shown herein for each cancer can be determined to identify, and/or stage, and/or provide a prognosis for the indicated cancer. In certain embodiments, methylation status of all of the genes shown for a particular cancer (forward and/or reverse strand) can be determined in a single multiplex PCR reaction.

It will be understood by those of skill in the art, that the methods of the present invention can be used to quantify methylated and unmethylated DNA in a sample from a subject. However, it is important to note that for various reasons, the quantity of unmethylated DNA in patients can fluctuate daily, and as such, it is less than optimal to compare methylated DNA quantity to a fluctuating reference point.

The inventive methods can be used to assess the methylation status of multiple genes, using very small quantities of DNA. A cumulative score of hypermethylation among multiple genes better distinguishes normal or benign from malignant tumors in bodily fluid samples as compared to the value of individual gene methylation markers.

It should be recognized that an amplification “primer pair” as the term is used herein requires what are commonly referred to as a forward primer and a reverse primer, which are selected using methods that are well known and routine and as described herein such that an amplification product can be generated therefrom.

In accordance with another embodiment of the present invention, it will be understood that the term “biological sample” or “biological fluid” includes, but is not limited to, any quantity of a substance from a living or formerly living patient or mammal. Such substances include, but are not limited to, blood, serum, plasma, urine, cells, organs, tissues, bone, bone marrow, synovial tissue, chondrocytes, synovial macrophages, endothelial cells, and skin. In some embodiments, the sample can be a FFPE sample. In one preferred embodiment, the sample is a fine needle aspirate of a sample of breast tissue suspected to be a cancer or tumor. In other embodiments, the sample can be a core biopsy sample of a suspect lesion in breast, colon, or lung tissue.

In an embodiment, the inventive methods are only being illustrated using primary breast cancer as an example. In breast cancer, samples can be collected from such tissue sources as ductal lavage and nipple aspirate fluid where the DNA amount is limiting, for example as little as about 50 to about 100 cells, as well as in larger samples, such as formalin-fixed paraffin-embedded sections of core biopsies.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Study Design. To ensure widespread applicability of the test, we requested equal numbers of ductal cancer and benign breast tissues from each of three geographical regions, U.S., China, and S. Africa. Formalin-fixed paraffin-embedded (FFPE) breast tissues (N=455) from these regions were randomly assigned to training (N=230) and test (N=225) case-control sets (FIG. 1) using the Excel RANDBETWEEN function. The randomization was carried out separately for each geographical region, and type of lesion to ensure balance (Table 1). Later, 23 invasive lobular carcinomas (ILC) were added to the test set to ensure inclusion of this significant pathological subset of breast carcinoma. Our study design was the following: First, 25 hypermethylated candidate gene markers were screened in the training cohort to identify a minimal marker panel of 10-genes using the predetermined criteria described below. This marker panel was evaluated to establish the methylation threshold best able to distinguish between benign and cancer samples. Due to limited available DNA, it was not possible to evaluate all candidate markers in all samples of the training cohort, so the final set of markers was measured in 210 of the 230 samples. Second, the marker panel was examined in a test cohort of 222 samples, which consisted of 199 samples with sufficient DNA and the 23 samples of ILC. We evaluated the utility of the assay methylation threshold (defined in the training cohort) for the novel gene panel. This study was approved by the Johns Hopkins Institutional Review Board.

TABLE 1
Patient Characteristics

| | Sample Sets | | | | | | | | |
|---|---|---|---|---|---|---|---|---|---|
| | Training (FFPE) | | | | Test (FFPE) | | | | Pilot (FNA) |
| | Region; Total | | | | | | | | |
| | U.S. | China | S. Africa | Total | U.S. | China | S Africa | Total | Total |
| Sample N; Total | 87 | 89 | 50 | 226 | 109 | 88 | 49 | 246 | 76 |
| Invasive Ductal | 29 | 29 | 13 | 71 | 29 | 30 | 13 | 72 | 24 |
| Carcinoma, N | | | | | | | | | |
| Receptor status | 11 | 19 | 8 | 38 | 3 | 18 | 5 | 26 | 16 |
| ER/PR+, HER2− | | | | | | | | | |
| ER/PR+, HER2+ | 2 | 2 | 3 | 7 | 1 | 3 | 3 | 7 | 2 |
| ER/PR−, HER2+ | 2 | 4 | 0 | 6 | 1 | 1 | 1 | 3 | 1 |
| ER/PR−, HER2− | 4 | 4 | 2 | 10 | 11 | 7 | 3 | 21 | 3 |
| Unknown | 10 | 0 | 0 | 10 | 13 | 1 | 1 | 15 | 2 |
| Age (in years) | 56 | 53 | 50 | 54 | 54 | 50 | 55 | 54 | 59 |
| Median | | | | | | | | | |
| Range | 25-87 | 23-66 | 32-74 | 23-87 | 25-70 | 35-75 | 34-88 | 25-88 | 30-87 |
| Invasive Lobular | — | — | — | — | 23 | — | — | 23 | 0 |
| Carcinoma, N | | | | | | | | | |
| Receptor status | — | — | — | — | 19 | — | — | 19 | — |
| ER/PR+ | | | | | | | | | |
| ER/PR− | — | — | — | — | 4 | — | — | 4 | — |
| Unknown | — | — | — | — | — | — | — | — | — |
| Age (in years) | — | — | — | — | 58 | — | — | 58 | — |
| Median | | | | | | | | | |
| Range | — | — | — | — | 38-81 | — | — | 38-81 | — |
| Ductal Carcinoma | 18 | 8 | 13 | 39 | 18 | 7 | 13 | 38 | 0 |
| In Situ, N | | | | | | | | | |
| Age (in years) | | | | | | | | | |
| Median | 58 | 54 | 59 | 56 | 56 | 53 | 46 | 53 | — |
| Range | 42-78 | 25-71 | 37-76 | 25-78 | 42-79 | 45-72 | 26-82 | 26-82 | — |
| Benign breast | 40 | 52 | 12 | 104 | 39 | 51 | 12 | 102 | 52 |
| disease, N | | | | | | | | | |
| Age (in years) | 51 | 46 | 39 | 47 | 52 | 48 | 37 | 47 | 41 |
| Median | | | | | | | | | |
| Range | 26-78 | 26-68 | 17-62 | 17-78 | 36-75 | 23-73 | 19-60 | 19-75 | 19-74 |
| Normal breast; N | — | — | 12 | 12 | — | — | 11 | 11 | 0 |
| Age (in years) | — | — | 45 | 45 | — | — | 45 | 45 | — |
| Median | | | | | | | | | |
| Range | — | — | 37-96 | 37-96 | — | — | 23-69 | 23-69 | — |

Patient Materials

DNA was extracted from FFPE tissues (N=472) obtained from Johns Hopkins Surgical Pathology, Renmin Hospital of Wuhan University, China, and National Health Laboratory Service, S. Africa, following review by a breast pathologist to confirm correct classification. Breast cancer samples included invasive ductal carcinoma (IDC), invasive lobular carcinoma (ILC), and ductal carcinoma in situ (DCIS). Two non-cancer groups were studied: normal breast and benign breast disease. The cancer FFPE blocks ranged in age from 2-28 years (median 4 years), and the benign/normal blocks ranged in age from 2-19 years (median 2 years). A large majority of our FFPE samples were 2-10 years old [cancer (73%), benign (89%)]. The immunohistochemical subtype information was available for estrogen/progesterone receptor (ER/PR) and HER2 among a subgroup of the cancer samples in the training and test sets (N=112). This information was used to determine if gene methylation correlated with tumor receptor status and geographic origin.

Assay Development, Marker selection and Training

QM-MSP was used in the training and test sets for marker selection and evaluation. Twenty-five individual markers were assayed using DNA from FFPE tissues in the training cohort. Marker selection criteria required first, that markers show considerably higher median methylation in cancer than in benign/normal tissue (P<0.002, based on the Mann-Whitney test). Secondly, to further minimize the risk of false positives, benign and normal samples were required to be methylated at lower levels and less frequently compared to cancer samples. Specifically, we calculated the 75th percentile of methylation separately in tumor and benign samples, discarding any markers in which the 75th percentile of normal methylation was high, or where the difference between normal and tumor was small (Table 2). The selected markers were then evaluated as a panel in the training set. QM-MSP values for the panel were expressed as Cumulative Methylation Index (CMI), the sum of percent methylation for each gene in the panel (22, 23). Using Receiver Operating Characteristic (ROC) curve analysis we identified the laboratory CMI threshold for the study that maximized specificity while maintaining sensitivity at 90%.

TABLE 2
Selection of marker gene-panel.
Individual Gene Marker Selection (25 markers)

| | AKR1B1 | | APC | | CCND2 | | COL6A2 | | HIST1H3C | |
|---|---|---|---|---|---|---|---|---|---|---|
| Tissue | Ca. | B | Ca. | B | Ca. | B | Ca. | B | Ca. | B |
| Number of Samples | 107 | 114 | 109 | 116 | 103 | 110 | 109 | 116 | 107 | 116 |
| % M per sample: | 0 | 0 | 0 | 0 | 0 | 0 | 0 | 0 | 0 | 0 |
| Minimum | | | | | | | | | | |
| 25th Percentile | 0 | 0 | 0 | 0 | 0 | 1 | 0 | 0 | 0 | 0 |
| 75th Percentile | 10 | 0 | 33 | 0 | 10 | 1 | 6 | 1 | 19 | 0 |
| Maximum | 62 | 4 | 90 | 24 | 74 | 14 | 82 | 22 | 82 | 3 |
| | HOXB4 | | RASGRF2 | | RASSF1 | | TMEFF2 | | ZNF671 | |
| Tissue | Ca. | B | Ca. | B | Ca. | B | Ca. | B | Ca. | B |
| Number of Samples | 107 | 113 | 102 | 112 | 108 | 114 | 103 | 113 | 107 | 112 |
| % M per sample: | 0 | 0 | 0 | 0 | 0 | 0 | 0 | 0 | 0 | 0 |
| Minimum | | | | | | | | | | |
| 25th Percentile | 0 | 0 | 1 | 0 | 0 | 0 | 0 | 0 | 0 | 0 |
| 75th Percentile | 5 | 0 | 26 | 2 | 27 | 2 | 11 | 0 | 8 | 1 |
| Maximum | 79 | 0 | 85 | 12 | 88 | 26 | 99 | 15 | 75 | 19 |

| | ARHGEF7 | | CBF2T3 | | CDKL2 | | cg18191418 | | EVI1 | | GAS7 | | GPX7 | | HIN | | MAL | | NEK9 | |
|---|---|---|---|---|---|---|---|---|---|---|---|---|---|---|---|---|---|---|---|---|
| Tissue | Ca. | B | Ca. | B | Ca. | B | Ca. | B | Ca. | B | Ca. | B | Ca. | B | Ca. | B | Ca. | B | Ca. | B |
| Number of Samples | 108 | 115 | 22 | 11 | 49 | 63 | 22 | 10 | 21 | 25 | 7 | 5 | 70 | 72 | 64 | 53 | 96 | 107 | 24 | 12 |
| % M per sample: | 0 | 0 | 0 | 0 | 0 | 1 | 0 | 0 | 0 | 0 | 0 | 0 | 0 | 0 | 0 | 0 | 0 | 0 | 0 | 0 |
| Minimum | | | | | | | | | | | | | | | | | | | | |
| 25th Percentile | 0 | 0 | 0 | 0 | 8 | 2 | 0 | 0 | 1 | 3 | 0 | 0 | 0 | 0 | 0 | 0 | 0 | 0 | 0 | 0 |
| 75th Percentile | 0 | 0 | 0 | 0 | 53 | 8 | 0 | 0 | 19 | 6 | 2 | 0 | 5 | 2 | 4 | 0 | 7 | 0 | 0 | 0 |
| Maximum | 84 | 5 | 8 | 0 | 68 | 44 | 6 | 0 | 86 | 24 | 3 | 0 | 94 | 20 | 37 | 69 | 69 | 5 | 0 | 0 |

| | | | | |
|---|---|---|---|---|
| RARB | TM6SF1 | TREX1 | TWIST1 | WNT1 |

| Tissue | Ca. | B | Ca. | B | Ca. | B | Ca. | B | Ca. | B |
|---|---|---|---|---|---|---|---|---|---|---|
| Number of Samples | 106 | 113 | 67 | 74 | 32 | 13 | 74 | 75 | 32 | 13 |
| % M per sample: | 0 | 0 | 0 | 0 | 0 | 0 | 0 | 0 | 0 | 0 |
| Minimum | | | | | | | | | | |
| 25th Percentile | 1 | 0 | 0 | 4 | 0 | 0 | 0 | 0 | 0 | 0 |
| 75th Percentile | 4 | 2 | 18 | 10 | 0 | 1 | 7 | 1 | 0 | 0 |
| Maximum | 98 | 7 | 84 | 35 | 10 | 4 | 42 | 4 | 23 | 0 |

Ca. = Cancer (IDC, DCIS), B = Benign/Normal; % M = Percent methylation of gene.

Percent methylation (minimum, 25th and 75th percentile, and maximum) is shown for each of 25 genes analyzed by QM-MSP in the training cohort of FFPE breast cancer and benign/normal samples. Sixteen markers showed considerably higher median methylation in cancer than in benign/normal (P<0.01, based on the Mann-Whitney test). To further minimize the risk of false positives, each remaining marker was examined to determine that levels of methylation in benign and normal samples were low. Specifically, the 75th percentile of methylation was calculated separately in tumor and benign samples, discarding any markers in which the 75th percentile of normal methylation was high, or where the difference between normal and tumor was small. The ten genes that were finally selected are denoted in red. Ca. cancer; B, benign; ns, not significant.

Marker Testing

The threshold and parameters defined in the training cohort for the marker panel using QM-MSP analysis was then tested in an independent test cohort (N=223) consisting of 66 IDC, 23 ILC, 30 DCIS, 91 benign and 10 normal breast FFPE tissues. Results were reported as sensitivity and specificity with exact confidence intervals.

Comparison of Methylation Based on Histologic Subtype of the Tumor and Region

For the four histologic subtypes of breast cancer, ER/PR+, ER/PR+ Her2+, ER/PR− HER2+, ER/PR− HER2− (triple-negative breast cancer, TNBC, differences between the four groups for cumulative methylation of the 10-gene marker panel, and methylation in individual markers was assessed by a three-way Kruskal-Wallis test.

For comparing samples in cohorts from the different geographic regions, both differences in cumulative methylation of the 10-gene marker panel, and methylation in individual markers between samples from the U.S., China, and South Africa were assessed by a three-way Kruskal-Wallis test. Mann-Whitney pairwise analysis was also performed to assess differences in individual gene methylation between two regions.

### Example 1

Selection of methylated gene markers

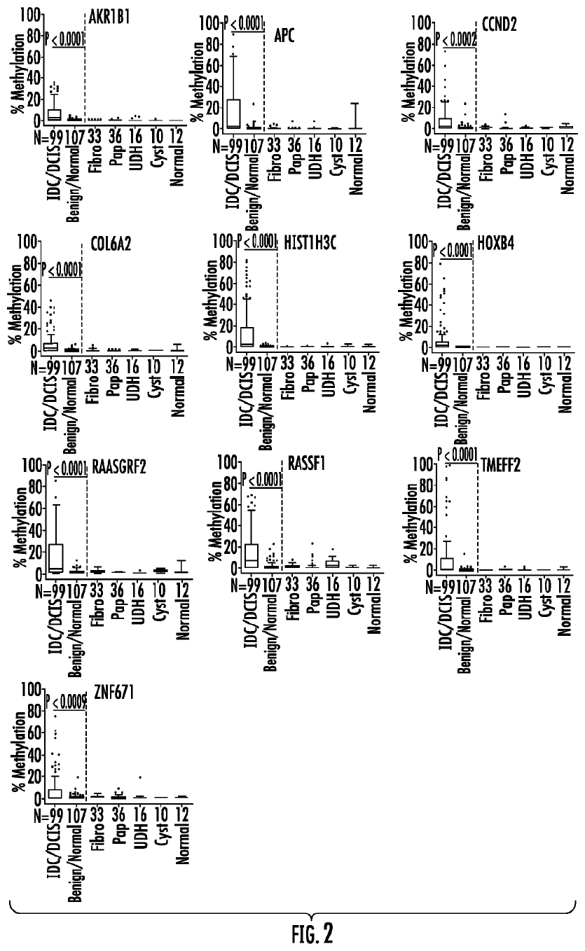

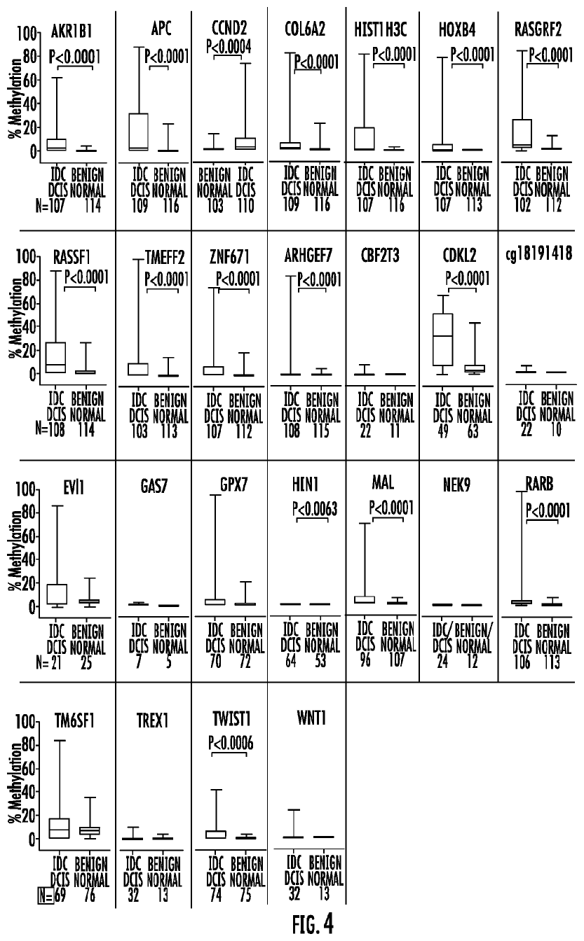

About 25 candidate gene markers were evaluated in the training set of 226 FFPE archival samples using QM-MSP to identify those genes with the highest sensitivity and specificity for cancer based on histopathology of the core biopsy (FIG. 1). Candidate gene markers were among those previously identified by the inventors as having frequent measurable hypermethylation in histopathological subtypes of ER/PR/HER2-positive and triple negative breast cancer (24,25). Nine of the 25 markers were discarded based on their inability to significantly distinguish between cancers (Invasive cancer/DCIS) and benign/normal breast tissues (FIG. 4). Each of the remaining 16 markers which showed significant differences between cancer and benign tissues (p<0.006 by Mann-Whitney) were screened individually for presence of high levels of methylation in the cancer samples, and in the same gene, for low levels of methylation in benign tissues (FIG. 4, Table 2). Among the 12 markers thus selected, the TWIST, MAL, CDKL2 and EVI1 gene markers were discarded based on poor assay performance. The 10 markers (p<0.003 by Mann-Whitney) in the panel were: AKR1B1, APC, CCND2, COL6A2, HIST1H3C, HOXB4, RASGRF2, RASSF1, TMEFF2, and ZNF671 (Table 2, FIG. 2).

### Example 2

Evaluation of Performance of a 10-Gene Marker Panel in Breast Cancer.

Using training set samples, Cumulative methylation for the 10-gene panel was higher in cancer compared to benign/normal samples as shown in the histogram (FIG. 3A) and in the box-plot (FIG. 3A, inset). ROC analyses of the training set established the laboratory threshold that provided the optimal specificity while retaining ≥90% sensitivity. A sensitivity of 90% (95% CI=82-95%), a specificity of 88% (95% CI=80-93%), and an AUC=0.948 (95% CI=0.914-0.976) was achieved in the training data at a threshold of 14.5 CMI units (FIG. 3A, inset).

### Example 3

Verification of the Performance of a 10-gene marker panel.

Parameters defined in the training set were locked for analysis in the independent test set of 220 cancer, benign and normal breast tissues using QM-MSP. We calculated the cumulative methylation in the test set for all 10 genes in the panel, shown as a histogram (FIG. 3B). The marker panel in the test set was significantly more methylated in IDC/ILC/DCIS compared to benign/normal tissues as shown in the box plot (P<0.0001; FIG. 3B, inset; Mann-Whitney). Using ROC analyses, the sensitivity achieved was 87% (95% CI=80-93%) and specificity was 88% (95% CI=80-94%) at a CMI threshold of 14.5 units (AUC=0.937, 95% CI=0.900-0.970; P<0.0001) (FIG. 3B, inset).

### Example 4

Methylation Frequency by Histologic Subtype and Region Histologic subtype. Using a laboratory threshold value of 14.5 CMI units, each of the subtypes had a similar percentage of tumors that scored positive by QM-MSP (86-100%; P=0.50 by Fisher's Exact, Table 3A) and no significant difference (P=0.077) in CMI of the 10-gene panel (FIG. 5A). However, between the four subtypes the individual markers showed a varying extent of methylation (Table 3B and FIG. 5A). For example, ZNF671 was significantly more methylated in ER/PR−, HER2−(TNBC) compared to ER/PR+, HER2− tumors (P<0.0001). For ER/PR−, HER2+ tumors the methylation of APC, RASGRF2, RASSF1, and TMEFF2 was higher than for other IHC subtypes (P values not adjusted)

TABLE 3A
Methylation by IHC Subtypes
Frequency of Methylation in Invasive Breast Cancer
by Histologic Subtype using the 10-marker Panel

| Histologic | | Not | % | |
|---|---|---|---|---|
| Subtype | Methylated* | Methylated | Methylated | P** |
| ER/PR+, HER2− | 56 | 6 | 90 | 0.50 |
| ER/PR+, HER2+ | 14 | 0 | 100 | |
| ER/PR−, HER2+ | 9 | 0 | 100 | |
| ER/PR−, HER2− | 19 | 3 | 86 | |

*Above the threshold of 14.5 cumulative methylation units
**Fisher Exact for 4-way comparison between histologic subtypes

Table 3B: Comparison of Subtypes

Comparison Between IHC Subtypes for Methylation Levels of Individual Markers

Regional Variation.

Cumulative methylation of the 10-gene panel did not differ significantly between the regions of U.S., China, and S. Africa (P=0.265 for benign/normal and P=0.474 for cancer, Kruskal-Wallis; FIG. 5B). However, for individual markers, regional differences were detected for CCND2, COL6A2, HIST1H3C and ZNF671 (Kruskal-Wallis P values=0.010, 0.003, 0.003, 0.043 respectively. AUC values showed modest differences between U.S. (AUC=0.941; 95% CI=0.890-0.983), China (AUC=0.901; 95% CI=0.813-0.970), and S. Africa (AUC=0.958; 95% CI 0.872-1.00) (FIG. 5B). Performance of the 10-gene panel (as measured by area under the ROC curve) was assessed separately in tissues from the U.S., China and Africa. The dashed line indicates our target sensitivity at 90%, and dots indicate the cutoff at 14.5 CMI units on each curve. Sensitivity, specificity and AUC values with 95% CI are shown in the table (FIG. 5C)

Collectively, the similarities observed in cumulative methylation in breast cancers suggested that the 10-gene marker panel embodiment performed well as a “pan breast cancer” detection tool across the main histologic subtypes of breast cancer and within the three distinct regional populations evaluated.

### Example 5

Evaluation of Performance of a 13-Gene Marker Panel in Colon Cancer.

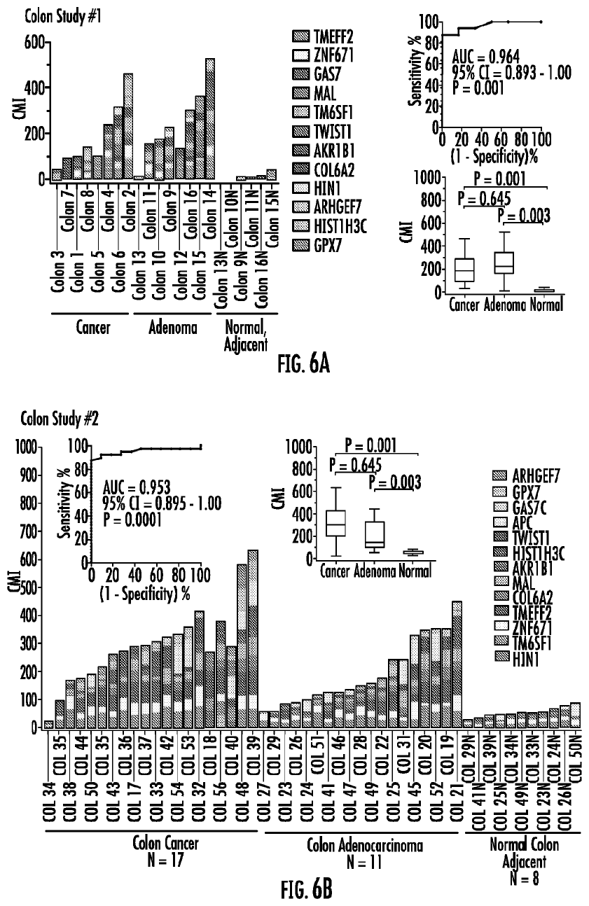

Cumulative methylation of a 13-gene marker panel was evaluated in colon cancer tissues (FIG. 6). Slides consisting of sections of formalin fixed paraffin embedded tissue were macrodissected, processed and QM-MSP was performed to determine the extent of DNA methylation within the sample. The histogram displays the cumulative methylation level of the panel, as well as the percent methylation of each gene (colored segment). Colon Study #1 (FIG. 6A) consisted of 8 cancers, 8 benign adenomas and 6 normal tissues located adjacent to cancer/adenoma. Colon Study #2 (FIG. 6B) consisted of 17 cancers, 11 adenomas and 9 normal adjacent tissues. ROC analyses were able to accurately distinguish between cancer/adenoma versus normal tissue (AUC 0.964 and 0.950, Study 1 and 2, respectively) and Mann-Whitney analyses showed significantly higher methylation in cancer/adenoma, compared to normal tissue. Since adenoma tissues are at high risk for colon cancer it is common practice to excise adenoma when observed during colonoscopy or colon surgery.

### Example 6

Evaluation of Performance of a 12-Gene Marker Panel in Lung Cancer.

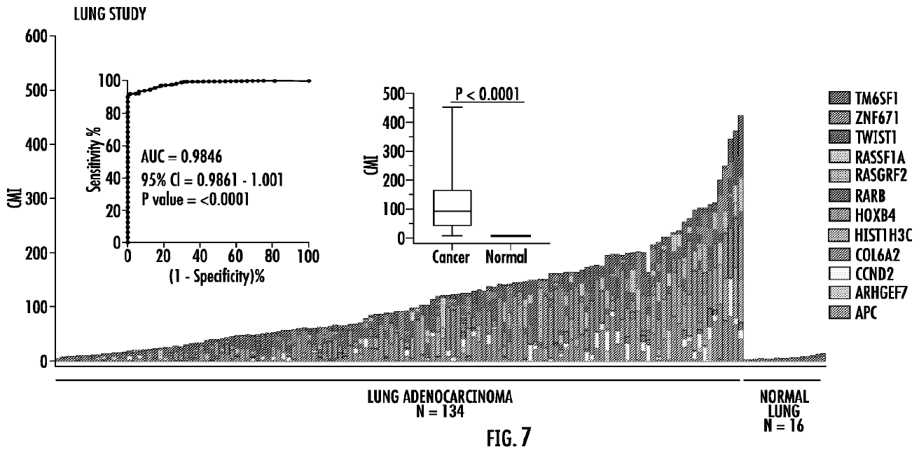

Cumulative methylation of a 12-gene marker panel was evaluated in lung cancer (FIG. 7). Slides of formalin fixed paraffin embedded tissue sections were scraped, processed and QM-MSP was performed to determine the extent of DNA methylation in the sample. The histogram displays the cumulative methylation level of the marker panel, as well as the percent methylation of each gene (colored segment) for 134 lung adenocarcinoma and 16 normal tissues adjacent to tumor. ROC analyses were able to accurately distinguish between adenocarcinoma and normal lung tissue (AUC 0.9846, P<0.0001) and Mann-Whitney analyses showed significantly higher methylation in adenocarcinoma compared to normal lung tissue (P<0.0001).

All references, including publications, patent applications, and patents, cited herein are hereby incorporated by reference to the same extent as if each reference were individually and specifically indicated to be incorporated by reference, and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Jemal A, Bray F, Center M M, Ferlay J, Ward E, Forman D. Global cancer statistics. C A Cancer J Clin 2011; 61(2):69-90 doi 10.3322/caac.20107.
2. Soerjomataram I, Lortet-Tieulent J, Parkin D M, Ferlay J, Mathers C, Forman D, et al. Global burden of cancer in 2008: a systematic analysis of disability-adjusted life-years in 12 world regions. Lancet 2012; 380(9856):1840-50 doi 10.1016/S0140-6736(12)60919-2.
3. Forouzanfar M H, Foreman K J, Delossantos A M, Lozano R, Lopez A D, Murray C J, et al. Breast and cervical cancer in 187 countries between 1980 and 2010: a systematic analysis. Lancet 2011; 378(9801):1461-84 doi 10.1016/S0140-6736(11)61351-2.
4. Shulman L N, Willett W, Sievers A, Knaul F M. Breast cancer in developing countries: opportunities for improved survival. J Oncol 2010; 2010:595167 doi 10.1155/2010/595167.
5. Ezzati M, Pearson-Stuttard J, Bennett J E, Mathers C D. Acting on non-communicable diseases in low- and middle-income tropical countries. Nature 2018; 559(7715):507-16 doi 10.1038/s41586-018-0306-9 10.1038/s41586-018-0306-9 [pii].
6. Plevritis S K, Munoz D, Kurian A W, Stout N K, Alagoz O, Near A M, et al. Association of Screening and Treatment With Breast Cancer Mortality by Molecular Subtype in U S Women, 2000-2012. Jama 2018; 319(2):154-64 doi 10.1001/jama.2017.19130.
7. Coldman A, Phillips N, Wilson C, Decker K, Chiarelli A M, Brisson J, et al. Pan-Canadian study of mammography screening and mortality from breast cancer. J Natl Cancer Inst 2014;106(11) doi 10.1093/jnci/dju261.
8. Cancer screening in the United States, 2017: A review of current American Cancer Society guidelines and current issues in cancer screening. Smith, R A et al. C A Cancer J Clin. 67:100-121 (2017).
9. Distelhorst S R, Cleary J F, Ganz P A, Bese N, Camacho-Rodriguez R, Cardoso F, et al. Optimisation of the continuum of supportive and palliative care for patients with breast cancer in low-income and middle-income countries: executive summary of the Breast Health Global Initiative, 2014. Lancet Oncol 2015;16(3):e137-47 doi 10.1016/S1470-2045(14)70457-7.
10. Yip C H, Smith R A, Anderson B O, Miller A B, Thomas D B, Ang E S, et al. Guideline implementation for breast healthcare in low- and middle-income countries: early detection resource allocation. Cancer 2008;113(8 Suppl):2244-56 doi 10.1002/cncr.23842.
11. Nelson A M, Milner D A, Rebbeck T R, Iliyasu Y. Oncologic Care and Pathology Resources in Africa: Survey and Recommendations. J Clin Oncol 2016; 34(1):20-6 doi 10.1200/JCO.2015.61.9767.
12. Unger-Saldana K, Infante-Castaneda C. Delay of medical care for symptomatic breast cancer: a literature review. Salud Publica Mex 2009; 51 Suppl 2:s270-85.
13. Baylin S B. DNA methylation and gene silencing in cancer. Nat Clin Pract Oncol 2005;2 Suppl 1:S4-11 doi 10.1038/ncponc0354.
14. Wittenberger T, Sleigh S, Reisel D, Zikan M, Wahl B, Alunni-Fabbroni M, et al. DNA methylation markers for early detection of women's cancer: promise and challenges. Epigenomics 2014; 6(3):311-27 doi 10.2217/epi.14.20.
15. Bu D, Lewis C M, Sarode V, Chen M, Ma X, Lazorwitz A M, et al. Identification of breast cancer DNA methylation markers optimized for fine-needle aspiration samples. Cancer Epidemiol Biomarkers Prev 2013; 22(12):2212-21 doi 10.1158/1055-9965.EPI-13-0208.
16. Euhus D, Bu D, Xie X J, Sarode V, Ashfaq R, Hunt K, et al. Tamoxifen downregulates ets oncogene family members ETV4 and ETV5 in benign breast tissue: implications for durable risk reduction. Cancer prevention research 2011; 4(11):1852-62 doi 10.1158/1940-6207.CAPR-11-0186.
17. Euhus D M, Bu D, Milchgrub S, Xie X J, Bian A, Leitch A M, et al. DNA methylation in benign breast epithelium in relation to age and breast cancer risk. Cancer Epidemiol Biomarkers Prev 2008; 17(5):1051-9 doi 10.1158/1055-9965.EPI-07-2582.
18. Lewis C M, Cler L R, Bu D W, Zochbauer-Muller S, Milchgrub S, Naftalis E Z, et al. Promoter hypermethylation in benign breast epithelium in relation to predicted breast cancer risk. Clinical cancer research: an official journal of the American Association for Cancer Research 2005; 11(1): 166-72.
19. Shan M, Yin H, Li J, Li X, Wang D, Su Y, et al. Detection of aberrant methylation of a six-gene panel in serum DNA for diagnosis of breast cancer. Oncotarget 2016; 7(14):18485-94 doi 10.18632/oncotarget. 7608.
20. van Hoesel A Q, Sato Y, Elashoff D A, Turner R R, Giuliano A E, Shamonki J M, et al. Assessment of DNA methylation status in early stages of breast cancer development. Br J Cancer 2013; 108(10):2033-8 doi 10.1038/bjc.2013.136.
21. Brooks J D, Cairns P, Shore R E, Klein C B, Wirgin I, Afanasyeva Y, et al. DNA methylation in pre-diagnostic serum samples of breast cancer cases: results of a nested case-control study. Cancer Epidemiol 2010; 34(6):717-23 doi 10.1016/j.canep.2010.05.006.
22. Sturgeon S R, Balasubramanian R, Schairer C, Muss H B, Ziegler R G, Arcaro K F. Detection of promoter methylation of tumor suppressor genes in serum DNA of breast cancer cases and benign breast disease controls. Epigenetics 2012; 7(11):1258-67 doi 10.4161/epi.22220.
23. Fackler M J, McVeigh M, Mehrotra J, Blum M A, Lange J, Lapides A, et al. Quantitative multiplex methylation-specific PCR assay for the detection of promoter hypermethylation in multiple genes in breast cancer. Cancer research 2004; 64(13):4442-52 doi 10.1158/0008-5472. CAN-03-3341.
24. Swift-Scanlan T, Vang R, Blackford A, Fackler M J, Sukumar S. Methylated genes in breast cancer: associations with clinical and histopathological features in a familial breast cancer cohort. Cancer biology & therapy 2011; 11(10):853-65.
25. Fackler M J, Lopez Bujanda Z, Umbricht C, Teo W W, Cho S, Zhang Z, et al. Novel methylated biomarkers and a robust assay to detect circulating tumor DNA in metastatic breast cancer. Cancer research 2014; 74(8):2160-70 doi 10.1158/0008-5472.CAN-13-339274/8/2160
26. Fackler M J, Umbricht C B, Williams D, Argani P, Cruz L A, Merino V F, et al. Genome-wide methylation analysis identifies genes specific to breast cancer hormone receptor status and risk of recurrence. Cancer research 2011;71 (19): 6195-207 doi 10.1158/0008-5472. CAN-11-1630.
27. Dedeurwaerder S, Desmedt C, Calonne E, Singhal S K, Haibe-Kains B, Defrance M, et al. DNA methylation profiling reveals a predominant immune component in breast cancers. EMBO Mol Med 2011; 3(12):726-41 doi 10.1002/emmm.201100801.
28. Fang F, Turcan S, Rimner A, Kaufman A, Giri D, Morris L G, et al. Breast cancer methylomes establish an epigenomic foundation for metastasis. Sci Transl Med 2011; 3(75):75ra25 doi 10.1126/scitranslmed. 3001875.
29. Urrutia R. KRAB-containing zinc-finger repressor proteins. Genome Biol 2003;4(10):231 doi 10.1186/gb-2003-4-10-231.
30. Stirzaker C, Zotenko E, Song J Z, Qu W, Nair S S, Locke W J, et al. Methylome sequencing in triple-negative breast cancer reveals distinct methylation clusters with prognostic value. Nat Commun 2015; 6:5899 doi 10.1038/ncomms6899.
31. Margolin J F, Friedman J R, Meyer W K, Vissing H, Thiesen H J, Rauscher F J, 3rd. Kruppel-associated boxes are potent transcriptional repression domains. Proc Natl Acad Sci USA 1994; 91(10):4509-13.

1. A method for detecting the presence of one, two, or more methylated gene regions in a biological sample of breast tissue from a subject suspected of having breast cancer comprising:
a) hybridizing nucleic acid obtained from the sample with two or more Q M-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671;
b) performing Q M-MSP on the breast tissue sample from a); and
c) detecting if any of the specific CpG regions of the one, two, or more genes of the breast tissue sample of a) are hypermethylated compared to the level of methylation of a normal/benign breast tissue sample.

2. A method for triaging a subject with one or more suspicious lesions in the breast into biopsy and pathological review comprising:
a) hybridizing nucleic acid obtained from a biological sample of breast tissue from the suspicious lesions in the breast of the subject with two or more Q M-MSP primer and probe sets specific for the genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671;
b) performing Q M-MSP on the breast tissue sample from a);
c) detecting if any of the specific CpG regions of the one, two, or more genes of the breast tissue of
a) are methylated compared to the level of methylation of normal/benign breast tissue sample; and
d) triaging the subject into biopsy of the suspicious lesion and pathological review when the specific CpG regions of one, two, or more of the genes of b) are hypermethylated compared to the level of methylation of a normal/benign breast tissue sample.

3. A method for monitoring disease or progression of disease in a subject having or suspected of having breast cancer comprising:
a) hybridizing nucleic acid obtained from a biological sample of breast tissue from the suspicious lesions in the breast of the subject with two or more Q M-MSP primer and probe sets specific for the genes selected from the group consisting of AKR1B1, APC, ARHGEF7, CCND2, CDKL2, COL6A2, EVIL GAS7C, GPX7, HIN1, HIST1H3C, HOXB4, MAL, RARB, RASGRF2, RASSF1, TM6SF1, TMEFF2, TWIST1, and ZNF671;
b) performing Q M-MSP on the breast tissue sample from a);
c) detecting if any of the specific CpG regions of the one, two, or more genes of the lymph node sample of a) are hypermethylated compared to the level of methylation of a previous sample of breast tissue from the subject; and
d) triaging the subject into further or different treatment when any of the specific CpG regions of the one, two, or more genes of the sample of breast tissue of a) are hypermethylated compared to the level of methylation of the previous sample of breast tissue from the subject.

4. A method for detecting disease recurrence in a subject undergoing treatment or having been treated for breast cancer comprising:
a) hybridizing nucleic acid obtained from a biological sample of breast tissue from the suspicious lesions in the breast of the subject with two, or more Q M-MSP primer and probe sets specific for the genes selected from the group consisting of AKR1B1, APC, ARHGEF7, CCND2, CDKL2, COL6A2, EVIL GAS7C, GPX7, HIN1, HIST1H3C, HOXB4, MAL, RARB, RASGRF2, RASSF1, TM6SF1, TMEFF2, TWIST1, and ZNF671;
b) performing Q M-MSP on the breast tissue sample from a);
c) detecting if any of the specific CpG regions of the one, two, or more genes of the biological sample of breast tissue of a) are hypermethylated compared to the level of methylation of a previous biological sample of breast tissue from the subject; and
d) triaging the subject into a change in treatment when any of the specific CpG regions of one, two, or more genes of the biological sample of breast tissue of a) are hypermethylated compared to the level of methylation of the previous biological sample of breast tissue from the subject.

5. The method of claim 1, wherein at step a) increased methylation is measured in 3, 4, 5, 6, 7, 8, 9, or 10, and up to 20 sets of different specific CpG regions of genes selected from the group consisting of AKR1B1, APC, ARHGEF7, CCND2, CDKL2, COL6A2, EVI1, GAS7C, GPX7, HIN1, HIST1H3C, HOXB4, MAL, RARB, RASGRF2, RASSF1, TM6SF1, TMEFF2, TWIST1, and ZNF671, as well as permutations thereof.

6. The method of claim 1, wherein the samples are from ductal lavage/ductoscopy fluids and cells, nipple fluids, fine needle aspirates, tissues and core biopsies.

7. A method for detecting the presence of two or more methylated gene regions in a biological sample from a suspicious colon lesion from a subject comprising:
a) hybridizing nucleic acid obtained from a sample of tissue, cells, stool, sigmoidoscopy or endoscopy-irrigation-derived cells, saliva, blood or urine from the subject with two or more Q M-MSP primers and probes specific for the genes selected from the group consisting of AKR1B1, APC, ARHGEF7, COL6A2, GAS7C, GPX7, HIN1, HIST1H3C, MAL, TM6SF1, TMEFF2, TWIST1, and ZNF671;
b) performing Q M-MSP on the sample from a); and
c) detecting if any of the specific CpG regions of the one, two, or more genes of a) are hypermethylated compared to the level of methylation of normal/benign colon tissue.

8-10. (canceled)

11. The method of claim 7, wherein the method detects increased methylation of at least 3 to about 13 different sets of specific CpG regions of genes selected from the group consisting of AKR1B1, APC, ARHGEF7, COL6A2, GAS7C, GPX7, HIN1, HIST1H3C, MAL, TM6SF1, TMEFF2, TWIST1, and ZNF671, as well as permutations thereof.

12. The method of claim 7, wherein the sample of the suspected colon lesion is taken from stool, blood, tissues, core biopsies, and suspicious colon polyps.

13. A method for detecting the presence of one, two, or more methylated gene regions in a biological sample from a suspicious lung lesion, including samples from oral gavage, saliva, as well as needle or core biopsies, blood, or urine, using a panel of methylated gene markers for lung cancer screening in subjects with suspicious lesions, comprising:
a) hybridizing nucleic acid obtained from a sample of the lesion from the subject with two or more Q M-MSP primer and probe sets specific for the genes selected from the group consisting of APC, ARHGEF7, CCND2, COL6A2, HIST1H3C, HOXB4, RARB, RASGRF2, RASSF1, TM6SF1, TWIST1, and ZNF671;
b) performing Q M-MSP on the sample from a); and
c) detecting if any of the specific CpG regions of the one, two, or more genes of a) are hypermethylated compared to the level of methylation of normal/benign lung tissue.

14-16. (canceled)

17. The method of claim 13, wherein the methods for detection of lung cancer can detect increased methylation of at least 3 to 12 different sets of specific CpG regions of genes selected from the group consisting of APC, ARHGEF7, CCND2, COL6A2, HIST1H3C, HOXB4, RARB, RASGRF2, RASSF1, TM6SF1, TWIST1, and ZNF671, as well as permutations thereof.

18. The methods of claim 13, wherein the biological sample is from oral gavage, saliva, needle or core biopsies, blood, urine, bronchoscopy, lung polyps, or sputum.

19. The methods of claim 1, wherein at step a) one, two, or more cMethDNA primer and probe sets are used.
